(19) United States
(12) Patent Application Publication
(10) Pub. No.: US 2026/0295184 A1
(43) Pub. Date: Oct. 1, 2026

(54) MEDICAL DEVICE AND NON-TRANSITORY STORAGE MEDIUM

(71) Applicant: FUKUDA DENSHI CO., LTD., Tokyo (JP)

(72) Inventors: Mai KOBAYASHI, Tokyo (JP); Ryotaro Hashimoto, Tokyo (JP); Tatsuya Yoneyama, Tokyo (JP)

(73) Assignee: FUKUDA DENSHI CO., LTD., Tokyo (JP)

(21) Appl. No.: 19/630,529

(22) Filed: Mar. 27, 2026

(30) Foreign Application Priority Data
Mar. 27, 2025 (JP) 2025-053686
Mar. 31, 2025 (JP) 2025-058362

(51) Int. Cl. A61M 16/00; A61M 16/08

(52) U.S. Cl. CPC A61M 16/024; A61M 16/08; A61M 2205/15; A61M 2205/3334

(57) ABSTRACT

Provided is medical device including a supplier configured to supply air to the airway of a patient at a treatment pressure and a processor. The processor is configured to change the treatment pressure by a first amount in a case in which a first condition relating to respiration disorder is satisfied while the treatment pressure is lower than a first threshold value, to change the treatment pressure by a second amount in a case in which a second condition relating to the respiration disorder is satisfied while the treatment pressure is equal to or higher than the first threshold value, and to change a setting for a pressure change parameter including at least one of the first amount, the second amount, the first condition, the second condition, or the first threshold value for a first operation mode of the medical device.

### BACKGROUND

The present disclosure relates to a medical device and a non-transitory storage medium that stores a program for making a computer execute a method of operating a medical device a program.

As a treatment method for the sleep apnea syndrome (SAS), there has been known the nasal continuous positive airway pressure (CPAP) treatment. The CPAP is a treatment method of continuously feeding air at an appropriate pressure to the airway of a patient through a nasal mask to prevent obstruction of the airway during the sleep, thereby suppressing an occurrence of the apnea and the hypopnea. In JP 2015-514465T, there is described chronologically changing a pressure level. As a sign of the occurrence of the apnea and the hypopnea, a snore sometimes occur, and, in JP 2012-5859A, there is described a technology of detecting the occurrence of the snore.

### SUMMARY

The patient possibly feels discomfort about the use of the CPAP device and stops the use thereof at patient's own decision. Some aspects of the present disclosure have an object to provide a technology for increasing continuity of treatment.

According to some embodiments, there is provided a medical device including a supplier configured to supply air to an airway of a patient at a treatment pressure and a processor. The processor is configured to change the treatment pressure by a first amount in a case in which a first condition relating to respiration disorder is satisfied while the treatment pressure is lower than a first threshold value, change the treatment pressure by a second amount in a case in which a second condition relating to the respiration disorder is satisfied while the treatment pressure is equal to or higher than the first threshold value, and change a setting for a pressure change parameter including at least one of the first amount, the second amount, the first condition, the second condition, or the first threshold value for a first operation mode of the medical device.

According to some embodiments, there is provided a medical device including a processor configured to detect that an analysis target portion of a respiratory waveform of a patient includes a high-frequency component that satisfies a predetermined condition and identify, on the basis of a determination criterion, a cause of the high-frequency component among a plurality of causes. The plurality of causes include a snore, and the determination criterion is based on whether or not an inspiration portion of the respiratory waveform includes the high-frequency component.

According to some embodiments, the continuity of the treatment can be increased.

### BRIEF DESCRIPTION OF THE DRAWINGS

FIG. 1 is a block diagram for illustrating a configuration example of a CPAP device according to some embodiments (first embodiment);

FIG. 2 is a flowchart for illustrating a change method for a treatment pressure according to some embodiments (first embodiment);

FIG. 3A is a schematic diagram for illustrating an example of the change in treatment pressure in a plurality of operation modes according to some embodiments (first embodiment);

FIG. 3B is a schematic diagram for illustrating an example of the change in treatment pressure in the plurality of operation modes according to some embodiments (first embodiment);

FIG. 3C is a schematic diagram for illustrating an example of the change in treatment pressure in the plurality of operation modes according to some embodiments (first embodiment);

FIG. 3D is a schematic diagram for illustrating an example of the change in treatment pressure in the plurality of operation modes according to some embodiments (first embodiment);

FIG. 4 is a flowchart for illustrating an example of a change method for a setting for pressure change parameters according to some embodiments (first embodiment);

FIG. 5 is a schematic diagram for illustrating an example of a screen which acquires a specification for the treatment parameters according to some embodiments (first embodiment);

FIG. 6 is a schematic diagram for illustrating an example of the change method for the setting for the pressure change parameters according to some embodiments (first embodiment);

FIG. 7A is a schematic diagram for illustrating an example of a change method for a setting for pressure increase parameters according to some embodiments (first embodiment);

FIG. 7B is a schematic diagram for illustrating an example of a change method for a setting for pressure reduction parameters according to some embodiments (first embodiment);

FIG. 8 is a schematic diagram for illustrating a graph provided to a medical doctor according to some embodiments (first embodiment);

FIG. 9 is a block diagram for illustrating a configuration example of a CPAP device according to some embodiments (second embodiment);

FIG. 10 is a flowchart for illustrating an example of a method of operating a CPAP device according to some embodiments (second embodiment);

FIG. 11 is a schematic diagram for illustrating an example of a method of determining various indices of a respiratory waveform according to some embodiments (second embodiment); and

FIG. 12 is a schematic diagram for illustrating an example of causes of a high-frequency component and processing corresponding thereto according to some embodiments (second embodiment).

### DETAILED DESCRIPTION OF THE PREFERRED EMBODIMENTS

A detailed description is now given of embodiments with reference to the accompanying drawings. Note that the following embodiments do not limit the disclosure according to claims and all of combinations of features described in the embodiments are not necessarily essential for the disclosure. Two or more features among the plurality of features described in the embodiments may freely be combined with one another. Moreover, the same or similar configurations are denoted by the same reference signs, and a redundant description thereof is omitted.

### First Embodiment

To a CPAP device, there are set, by a vendor in advance, values of parameters which change a treatment pressure such that the treatment pressure is optimal for treatment. However, when the treatment pressure is too high, a patient possibly feels discomfort about the use of the CPAP device and stops the use thereof at patient's own decision. The present embodiment makes an appropriate setting for pressure change parameters, thereby increasing continuity of the treatment while the patient does not feel discomfort about the use of the CPAP device.

### <Configuration of CPAP Device>

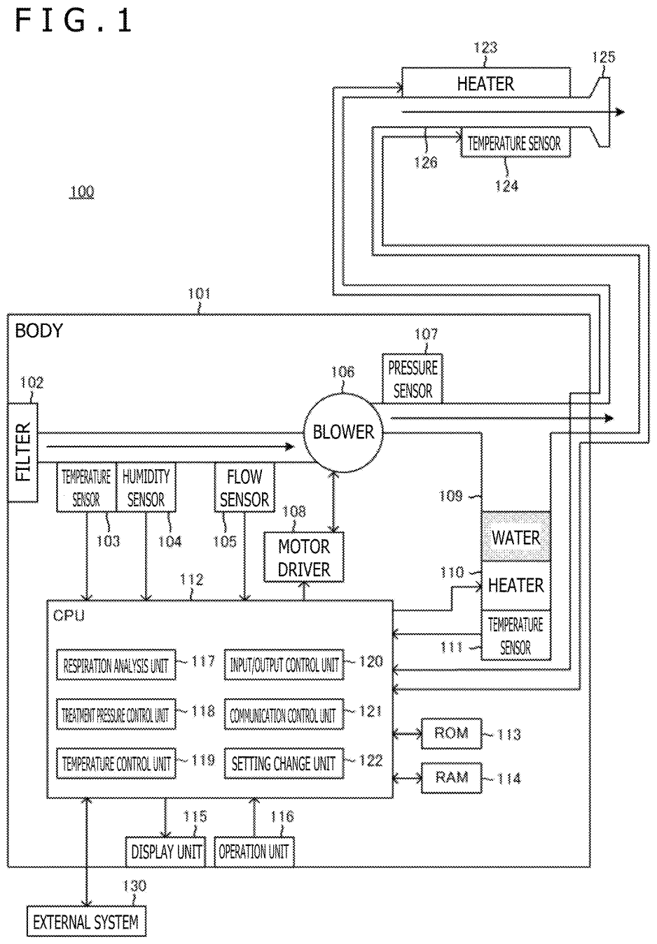

With reference to FIG. 1, a description is now given of a configuration example of a continuous positive airway pressure device (hereinafter referred to as the CPAP device) 100 according to some embodiments. The CPAP device 100 is an example of a medical device which treats the SAS. The medical device is a device used for the medicine. For example, the medical device is a device used for surgery, treatment, or diagnosis. The CPAP device 100 includes a body 101, a mask 125, and a tube 126 which connects the body 101 and the mask 125 to each other. An operation of the CPAP device 100 is implemented by a central processing unit (CPU) 112 reading a program stored in a read only memory (ROM) 113 into a random access memory (RAM) 114 and then executing the program. A device including a processor such as the CPU 112 and a memory such as the ROM 113 and the RAM 114 as described above may be considered as a computer. Note that functional blocks 117 to 122 illustrated in the CPU 112 are principal functions which are among the various functions implemented by the CPU 112 executing the program and which are expressed schematically. Thus, an operation described as being performed by each of the functional blocks 117 to 122 is, in practice, implemented by the CPU 112 executing the program. In place thereof, one or more functional blocks may be implemented by a hardware circuit other than the CPU 112.

First, a description is now given of components that are present in a flow passage of air. A filter 102 is provided at an inlet opening of the air, and removes pollens, bacteria, dusts, and the like. A temperature sensor 103 measures a temperature of the air which has flown in. A measurement value obtained by the temperature sensor 103 is supplied to a temperature control unit 119. A humidity sensor 104 measures humidity of the air which has flown in. A measurement value obtained by the humidity sensor 104 is supplied to the temperature control unit 119.

A flow (differential pressure) sensor 105 (hereinafter simply referred to as a flow sensor 105) is, for example, a flow rate sensor of a differential pressure type, and measures the flow rate of the air in the flow passage on the basis of a pressure difference between an upstream side and a downstream side. Here, it is assumed that there is acquired a positive measurement value in a case in which the pressure on the upstream side is higher than the pressure on the downstream side and there is acquired a negative measurement value in a case in which the pressure on the downstream side is higher than the pressure on the upstream side. Thus, it is possible to also recognize a flow direction of the air in the flow passage from the measurement value of the flow sensor 105. The measurement value of the flow sensor 105 is supplied to a respiration analysis unit 117.

A blower 106 internally includes an impeller and a motor that drives the impeller. A treatment pressure control unit 118 controls a rotation speed of the motor via a motor driver 108. A flow rate and a supply pressure of the air supplied to the airway of the patient can thereby be adjusted.

A pressure sensor 107 is provided downstream of the blower 106 in the flow passage, and measures a pressure in the flow passage. A measurement value of the pressure sensor 107 is supplied to the treatment pressure control unit 118. The treatment pressure control unit 118 controls the supply pressure while assuming that the air is supplied to the airway of the patient at the pressure measured by the pressure sensor 107.

A humidifier 109 includes a water storage tank, and humidifies the air supplied to the tube 126. Here, the temperature control unit 119 controls a temperature of a heater 110 provided to the humidifier 109, thereby controlling an amount of the water vaporized from the water storage tank, that is, a degree of the humidification. A temperature sensor 111 measures a temperature of the heater 110, and supplies the measured temperature to the temperature control unit 119. In the present embodiment, the heater 110 is used for the humidifier 109 to humidify the air supplied to the airway of the patient as well as executing temperature adjustment. Note that the temperature adjustment for the air can be implemented through another method such as using, for example, a heater 123 provided to the tube 126. In a case in which the humidification and the temperature adjustment are executed independently of each other, the heater 110 and the temperature sensor 111 may not be provided to the humidifier 109. Moreover, for example, the air may be blown to a water surface in the water storage tank, or the flow passage may be disposed in such a manner as to pass through the water.

The tube 126 connects the body 101 and the mask 125 to each other. The tube 126 has an elastic property and a bending property in order to be able to easily follow a movement of the mask 125. A temperature sensor 124 which measures a temperature of the air supplied to the airway of the patient is provided to the tube 126. A measurement value of the temperature sensor 124 is supplied to the temperature control unit 119.

The mask 125 has such a size and a shape as to cover the nose and the mouth of the patient, and is worn by the patient through use of a string or band adjustable in length. For example, a supply unit which supplies the air to the airway of the patient at the treatment pressure includes the blower 106, the mask 125, and the tube 126.

A display unit 115 is, for example, a display provided to a housing of the body 101, and displays a message relating to handling of the CPAP device 100, various menu screens used to for setting for the CPAP device 100 and the like, measurement values of various sensors, and the like. The display on the display unit 115 is controlled by an input/output control unit 120.

An operation unit 116 is a general name of input devices operable by a user, for example, buttons and switches provided to the housing of the body 101. In a case in which the display unit 115 is a touch display, the display unit 115 and the operation unit 116 are integrally formed. An operation on the operation unit 116 is detected by the input/output control unit 120, and the CPU 112 executes an operation corresponding to the detected operation.

The respiration analysis unit 117 detects an occurrence of an event defined in advance, on the basis of the flow rate measured by the flow sensor 105. When detecting the occurrence of the event defined in advance, the respiration analysis unit 117 notifies the treatment pressure control unit 118 of the occurrence of the event. In the present embodiment, the respiration analysis unit 117 may be able to detect an occurrence of each of a snore, flow limitation, apnea and hypopnea, and Cheyne-Stokes respiration in the patient. Further, the respiration analysis unit 117 may be able to detect a leak amount of the air from the mask 125.

The treatment pressure control unit 118 controls, on the basis of the measurement value of the pressure sensor 107, an operation of the blower 106 such that the air is supplied to the airway of the patient at the treatment pressure. Moreover, the treatment pressure control unit 118 controls a supply air pressure in response to the notification from the respiration analysis unit 117. The treatment pressure control unit 118 provides, to the motor driver 108, for example, a duty ratio of a pulse voltage applied to the motor, to control a rotation speed of the impeller of the blower 106, thereby controlling the supply air pressure. The treatment pressure control unit 118 notifies the temperature control unit 119 of the supply air pressure set currently.

The temperature control unit 119 controls the operation of the heater 110 according to the measurement values of the temperature sensor 103, the humidity sensor 104, the temperature sensor 111, and the temperature sensor 124 and the supply air pressure the notification of which is given from the treatment pressure control unit 118, thereby controlling the temperature and the humidity of the air supplied to the airway of the patient. The temperature and the humidity of the air supplied to the airway of the patient may be a temperature and humidity set by the user via the operation unit 116. The temperature control unit 119 is notified of the user setting made via the operation unit 116, via, for example, the input/output control unit 120. The supply air pressure is taken into consideration because a degree of an increase in temperature is low in a case in which the flow rate is high compared with a case in which the flow rate is low even when the temperature of the heater 110 is constant.

A communication control unit 121 executes processing relating to communication between the body 101 and an external system 130. The communication control unit 121 is compliant with, for example, one or more publicly-known wireless and/or wired communication standards and is able to execute the communication with the external system 130. The external system 130 may be, for example, a management system for examination/treatment data in a hospital or a remote management system for the CPAP device 100. A medical system includes the CPAP device 100 and the external system 130.

A setting change unit 122 changes a setting for pressure increase parameters of the treatment pressure. A specific example of the pressure increase parameters is described later. The setting change unit 122 may change the setting for the changed pressure increase parameters according to a specification by the user (for example, a medical doctor). In place thereof or in addition thereto, the setting change unit 122 may automatically change the setting for the pressure increase parameters in a case in which a specific condition is satisfied.

### <Method of Changing Treatment Pressure>

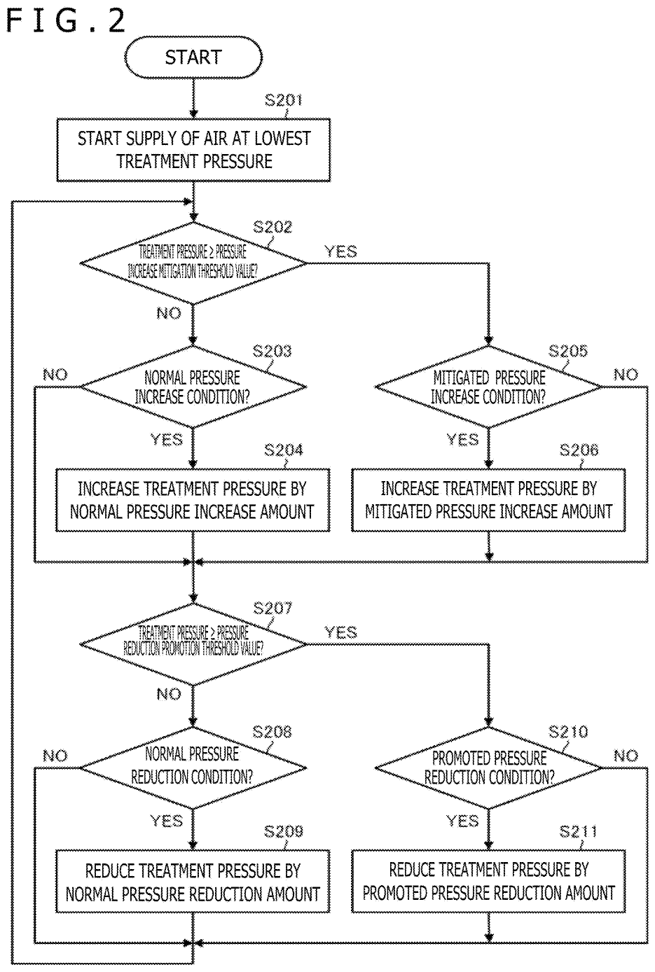

With reference to FIG. 2, a description is now given of a method of changing the treatment pressure of the CPAP device 100. The CPAP device 100 switches a type of increasing the treatment pressure, according to whether the current treatment pressure is higher than or lower than a specific threshold value (referred to as a pressure increase mitigation threshold value (first threshold value)), in order to reduce such possibilities that the patient awakes due to a sense of discomfort caused by a drastic increase in treatment pressure and that the aerophagy occurs in the patient. Specifically, the CPAP device 100 slowly increases the treatment pressure in a case in which the current treatment pressure is higher than the pressure increase mitigation threshold value, compared with a case in which the current treatment pressure is lower than the pressure increase mitigation threshold value. Moreover, the CPAP device 100 reduces the treatment pressure in response to such a state that the a respiration disorder event is not detected for a predetermined period of time after the CPAP device 100 increases the treatment pressure. When there continues the state in which the treatment pressure is high even when the respiration disorder event is not detected, there is such a possibility that the patient awakes due to the sense of discomfort or the aerophagy occurs. Thus, the CPAP device 100 switches a type of reducing the treatment pressure according to whether the current treatment pressure is higher than or lower than a specific threshold value (referred to as a pressure reduction promotion threshold value (third threshold value)). Specifically, the CPAP device 100 quickly reduces the treatment pressure in a case in which the current treatment pressure is higher than the pressure reduction promotion threshold value, compared with a case in which the current treatment pressure is lower than the pressure reduction promotion threshold value.

Each step of the method of FIG. 2 may be executed by the CPU 112 (for example, the treatment pressure control unit 118 thereof). Specifically, each step is executed by the CPU 112 executing the program read into the RAM 114. In place thereof, at least some of the steps of FIG. 2 may be executed by a dedicated circuit such as an application-specific integrated circuit (ASIC). The method of FIG. 2 may be started in response to such a state that start of an operation (for example, a treatment operation during the sleep) of the CPAP device 100 is instructed by the patient, may be started through, as a trigger, the CPAP device 100 detecting such a state that the patient falls asleep, or may be started through another event as a trigger. The method of FIG. 2 may be ended in response to such a state that end of the operation (for example, the treatment operation during the sleep) of the CPAP device 100 is instructed by the patient, may be ended through, as a trigger, the CPAP device 100 detecting such a state that the patient awakes, or may be ended through another event as a trigger.

The value measured by the flow sensor 105 is continued to be supplied to the CPU 112 (for example, the respiration analysis unit 117 thereof) during the execution of the method of FIG. 2. As described above, the value measured by the flow sensor 105 indicates the flow rate of the respiration of the patient. The CPU 112 samples the flow rate supplied by the flow sensor 105, at a predetermined sampling interval (for example, 2 ms), and stores, as flow rate data, the flow rate at each time in the RAM 114. This flow rate data indicates the flow rate of the respiration of the patient as a time series. The CPU 112 analyzes this flow rate data, thereby detecting the occurrence of the respiration disorder event in the patient. The respiration disorder event being a detection target includes at least one of the snore, the flow limitation, the apnea and the hypopnea, or the Cheyne-Stokes respiration.

In S201, the CPU 112 starts the supply of the air to the airway of the patient at the lowest treatment pressure. The lowest treatment pressure (for example, 4 cmH₂O) is set in advance by the medical doctor, a vendor of the CPAP device 100, or the like, and is stored in the RAM 114.

After the supply of the air is started in S201, the CPU 112 determines, in S202, whether or not the current treatment pressure is equal to or higher than the pressure increase mitigation threshold value. The pressure increase mitigation threshold value is a threshold value to be compared with the current treatment pressure in order to switch the type of the pressure increase processing. In a case in which the CPU 112 determines that the current treatment pressure is equal to or higher than the pressure increase mitigation threshold value (“YES” in S202), the CPU 112 causes the processing to transition to S205, otherwise (“NO” in S202) the CPU 112 causes the processing to transition to S203. The pressure increase mitigation threshold value (for example, 10 cmH₂O) is set in advance by the medical doctor, the vendor of the CPAP device 100, or the like, and is stored in the RAM 114. In the example of FIG. 2, in a case in which the current treatment pressure is equal to the pressure increase mitigation threshold value, the determination of “YES” is made in S202. In place thereof, in the case in which the current treatment pressure is equal to the pressure increase mitigation threshold value, a determination of “NO” may be made in S202. Further, in the case in which the current treatment pressure is equal to the pressure increase mitigation threshold value, the CPU 112 may execute both S203 and S205.

In S203, the CPU 112 determines whether or not a normal pressure increase condition (first condition) is satisfied. In a case in which the CPU 112 determines that the normal pressure increase condition is satisfied (“YES” in S203), the CPU 112 executes S204 and then causes the processing to transition to S207, otherwise (“NO” in S203) the CPU 112 causes the processing to transition to S207. The normal pressure increase condition is a condition for increasing the treatment pressure while the current treatment pressure is lower than the pressure increase mitigation threshold value. The normal pressure increase condition is set in advance by the medical doctor, the vendor of the CPAP device 100, or the like, and is stored in the RAM 114. The normal pressure increase condition may include such a condition that a predetermined number of times of a respiration disorder event have been detected from the start of the supply of the air at the current treatment pressure.

In a case in which the CPU 112 determines that the normal pressure increase condition is satisfied, the CPU 112 increases the treatment pressure by a normal pressure increase amount (first amount) in S204. The normal pressure increase amount is an amount by which the treatment pressure is increased while the current treatment pressure is lower than the pressure increase mitigation threshold value. The normal pressure increase amount (for example, 1 cmH₂O) is set in advance by the medical doctor, the vendor of the CPAP device 100, or the like, and is stored in the RAM 114. The pressure increase processing through use of the normal pressure increase condition and the normal pressure increase amount as that executed in S203 and S204 is referred to as normal pressure increase processing.

In S205, the CPU 112 determines whether or not the mitigated pressure increase condition (second condition) is satisfied. In a case in which the CPU 112 determines that the mitigated pressure increase condition is satisfied (“YES” in S205), the CPU 112 executes S206 and then causes the processing to transition to S207, otherwise (“NO” in S205) the CPU 112 causes the processing to transition to S207. The mitigated pressure increase condition is a condition for increasing the treatment pressure while the current treatment pressure is equal to or higher than the pressure increase mitigation threshold value. The mitigated pressure increase condition is set in advance by the medical doctor, the vendor of the CPAP device 100, or the like, and is stored in the RAM 114. The mitigated pressure increase condition may include such a condition that a predetermined number of times of the respiration disorder event have been detected from the start of the supply of the air at the current treatment pressure.

In a case in which the CPU 112 determines that the mitigated pressure increase condition is satisfied, the CPU 112 increases the treatment pressure by a mitigated pressure increase amount (second amount) in S206. The mitigated pressure increase amount is an amount by which the treatment pressure is increased while the current treatment pressure is equal to or higher than the pressure increase mitigation threshold value. In S206, the CPU 112 avoids such a state that the treatment pressure exceeds the highest treatment pressure. For example, in a case in which the treatment pressure would exceed the highest treatment pressure if the treatment pressure is increased by the mitigated pressure increase amount, the CPU 112 may not increase the treatment pressure or may increase the treatment pressure to the highest treatment pressure. The mitigated pressure increase amount (for example, 0.5 cmH₂O) and the highest treatment pressure (for example, 20 cmH₂O) are set in advance by the medical doctor, the vendor of the CPAP device 100, or the like, and are stored in the RAM 114. The pressure increase processing through use of the mitigated pressure increase condition and the mitigated pressure increase amount as that executed in S205 and S206 is referred to as mitigated pressure increase processing. The CPU 112 may stop the increase in treatment pressure even before the treatment pressure reaches the highest treatment pressure. For example, in a case in which the detected respiration disorder event is the central apnea, the apnea may not be dissolved even when the treatment pressure is increased, only obstructing the sleep of the patient. Moreover, also in a case in which apnea of the patient is suspected as central as a result of a diagnosis by the medical doctor, there is a case in which it is preferred that the unnecessary increase in treatment pressure be avoided. Thus, in such cases, the CPU 112 may set the mitigated pressure increase amount to zero even in a case in which the treatment pressure does not reach the highest treatment pressure.

The normal pressure increase condition, the normal pressure increase amount, the mitigated pressure increase condition, and the mitigated pressure increase amount are set such that the treatment pressure more slowly increases than in the normal pressure increase processing. For example, the normal pressure increase condition and the mitigated pressure increase condition may be the same as each other, and the mitigated pressure increase amount may be smaller than the normal pressure increase amount (for example, the mitigated pressure increase amount is a half of the normal pressure increase amount). In place thereof, the normal pressure increase amount and the mitigated pressure increase amount may be the same as each other, and the mitigated pressure increase condition may be less likely to be satisfied than the normal pressure increase condition. The state in which the mitigated pressure increase condition is less likely to be satisfied than the normal pressure increase condition means such a state that the mitigated pressure increase condition is a sufficient condition for the normal pressure increase but is not a necessary condition thereof. For example, a required number of times of the respiration disorder event to be detected to satisfy the mitigated pressure increase condition may be more than a required number of times of the respiration disorder event to be detected to satisfy the normal pressure increase condition. Further, the mitigated pressure increase amount may be smaller than the normal pressure increase amount, and the mitigated pressure increase condition may be less likely to be satisfied than the normal pressure increase condition.

In S207, the CPU 112 determines whether or not the current treatment pressure is equal to or higher than the pressure reduction promotion threshold value. The pressure reduction promotion threshold value is a threshold value to be compared with the current treatment pressure in order to switch the type of the pressure reduction processing. In a case in which the CPU 112 determines that the current treatment pressure is equal to or higher than the pressure reduction promotion threshold value (“YES” in S207), the CPU 112 causes the processing to transition to S210, otherwise (“NO” in S207) the CPU 112 causes the processing to transition to S208. The pressure reduction promotion threshold value (for example, 10 cmH₂O) is set in advance by the medical doctor, the vendor of the CPAP device 100, or the like, and is stored in the RAM 114. In the example of FIG. 2, in a case in which the current treatment pressure is equal to the pressure reduction promotion threshold value, the determination of “YES” is made in S207. In place thereof, in the case in which the current treatment pressure is equal to the pressure reduction promotion threshold value, the determination of “NO” may be made in S207. Further, in the case in which the current treatment pressure is equal to the pressure reduction promotion threshold value, the CPU 112 may execute both S208 and S210.

In S208, the CPU 112 determines whether or not a normal pressure reduction condition (fourth condition) is satisfied. In a case in which the CPU 112 determines that the normal pressure reduction condition is satisfied (“YES” in S208), the CPU 112 executes S209 and then causes the processing to transition to S202, otherwise (“NO” in S208) the CPU 112 causes the processing to transition to S202. The normal pressure reduction condition is a condition for reducing the treatment pressure while the current treatment pressure is lower than the pressure reduction promotion threshold value. The normal pressure reduction condition is set in advance by the medical doctor, the vendor of the CPAP device 100, or the like, and is stored in the RAM 114. The normal pressure reduction condition may include such a condition that the respiration disorder event has not been detected for a predetermined period of time from the start of the supply of the air at the current treatment pressure.

In a case in which the CPU 112 determines that the normal pressure reduction condition is satisfied, the CPU 112 reduces the treatment pressure by a normal pressure reduction amount (fourth amount) in S209. The normal pressure reduction amount is an amount by which the treatment pressure is reduced while the current treatment pressure is lower than the pressure reduction promotion threshold value. In S209, the CPU 112 avoids such a state that the treatment pressure falls below the lowest treatment pressure. For example, in a case in which the treatment pressure would fall below the lowest treatment pressure if the treatment pressure is reduced by the normal pressure reduction amount, the CPU 112 may not reduce the treatment pressure or may reduce the treatment pressure to the lowest treatment pressure. The normal pressure reduction amount (for example, 0.5 cmH₂O) and the lowest treatment pressure (for example, 4 cmH₂O) are set in advance by the medical doctor, the vendor of the CPAP device 100, or the like, and are stored in the RAM 114. The pressure reduction processing through use of the normal pressure reduction condition and the normal pressure reduction amount as that executed in S208 and S209 is referred to as normal pressure reduction processing.

In S210, the CPU 112 determines whether or not a promoted pressure reduction condition (fifth condition) is satisfied. In a case in which the CPU 112 determines that the promoted pressure reduction condition is satisfied (“YES” in S210), the CPU 112 executes S211 and then causes the processing to transition to S202, otherwise (“NO” in S210) the CPU 112 causes the processing to transition to S202. The promoted pressure reduction condition is a condition for reducing the treatment pressure while the current treatment pressure is equal to or higher than the pressure reduction promotion threshold value. The promoted pressure reduction condition is set in advance by the medical doctor, the vendor of the CPAP device 100, or the like, and is stored in the RAM 114. The promoted pressure reduction condition may include such a condition that a predetermined number of times of the respiration disorder event have been detected from the start of the supply of the air at the current treatment pressure.

In a case in which the CPU 112 determines that the promoted pressure reduction condition is satisfied, the CPU 112 reduces the treatment pressure by a promoted pressure reduction amount (fifth amount) in S211. The promoted pressure reduction amount is an amount by which the treatment pressure is reduced while the current treatment pressure is equal to or higher than the pressure reduction promotion threshold value. The promoted pressure reduction amount (for example, 1 cmH₂O) is set in advance by the medical doctor, the vendor of the CPAP device 100, or the like, and is stored in the RAM 114. The pressure reduction processing through use of the promoted pressure reduction condition and the promoted pressure reduction amount as that executed in S210 and S211 is referred to as promoted pressure reduction processing.

The normal pressure reduction condition, the normal pressure reduction amount, the promoted pressure reduction condition, and the promoted pressure reduction amount are set such that the treatment pressure more quickly decreases in the promoted pressure reduction processing than in the normal pressure reduction processing. For example, the normal pressure reduction condition and the promoted pressure reduction condition may be the same as each other, and the promoted pressure reduction amount may be larger than the normal pressure reduction amount (for example, the promoted pressure reduction amount is twice the normal pressure reduction amount). In place thereof, the normal pressure reduction amount and the promoted pressure reduction amount may be the same as each other, and the promoted pressure reduction condition may be more likely to be satisfied than the normal pressure reduction condition. The state in which the promoted pressure reduction condition is more likely to be satisfied than the normal pressure reduction condition means such a state that the promoted pressure reduction condition is a necessary condition of the normal pressure reduction but is not a sufficient condition thereof. For example, a required number of times of the respiration disorder event to be detected to satisfy the promoted pressure reduction condition may be less than a required number of times of the respiration disorder event to be detected to satisfy the normal pressure reduction condition. Further, the promoted pressure reduction amount may be larger than the normal pressure reduction amount, and the promoted pressure reduction condition may be more likely to be satisfied than the normal pressure reduction condition.

In the method of FIG. 2 described above, the CPAP device 100 compares the current treatment pressure with the one threshold value (pressure increase mitigation threshold value), thereby selecting any one of the two types of pressure increase processing (normal pressure increase processing and mitigated pressure increase processing), and then executes the selected processing. In place thereof, the CPAP device 100 may compare the current treatment pressure with two or more threshold values, thereby selecting any one of two or more types of pressure increase processing, and may then execute the selected processing. The two or more types of pressure increase processing may include, in addition to the above-described normal pressure increase processing and mitigated pressure increase processing, processing of increasing the treatment pressure more quickly than the normal pressure increase processing.

For example, the sleep of the patient is light at a treatment start time of the CPAP device 100, and hence, there is a risk that the patient may awake when the treatment pressure drastically increases from the lowest treatment pressure. Thus, the CPAP device 100 executes the mitigated pressure increase processing while the current treatment pressure is of the value close to the lowest treatment pressure. On the other hand, after the patient sufficiently deeply sleeps, a plurality of times of the respiration disorder occur in the patient, and the treatment pressure increases by a certain amount, a possibility that the patient awakes is low, and hence, the CPAP device 100 executes the normal pressure increase processing.

Specifically, while the current treatment pressure is equal to or higher than the lowest treatment pressure but lower than a lower pressure increase mitigation threshold value (second threshold value), the CPU 112 executes lower mitigated pressure increase processing. The CPU 112 executes the normal pressure increase processing while the current treatment pressure is equal to or higher than the lower pressure increase mitigation threshold value but lower than an upper pressure increase mitigation threshold value (first threshold value). In this example, in the case in which the current treatment pressure is equal to the lower pressure increase mitigation threshold value, the CPU 112 does not execute the lower mitigated pressure increase processing but executes the normal pressure increase processing. In place thereof, in the case in which the current treatment pressure is equal to the lower pressure increase mitigation threshold value, the CPU 112 may not execute the normal pressure increase processing but execute the lower mitigated pressure increase processing. The same applies to processing in a case in which the treatment pressure is equal to another threshold value described below. The CPU 112 executes upper mitigated pressure increase processing while the current treatment pressure is equal to or higher than the upper pressure increase mitigation threshold value but equal to or lower than the highest treatment pressure. The lower pressure increase mitigation threshold value and the upper pressure increase mitigation threshold value are each a threshold value to be compared with the current treatment pressure in order to switch the type of the pressure increase processing. The lower pressure increase mitigation threshold value is smaller than the upper pressure increase mitigation threshold value. The lower mitigated pressure increase processing and the upper mitigated pressure increase processing are each processing of increasing the treatment pressure more slowly than the normal processing as in the mitigated pressure increase processing described before. The mitigated pressure increase condition (third condition) in the lower mitigated pressure increase processing may be the same as the mitigated pressure increase condition (second condition) in the upper mitigated pressure increase processing, or may be different therefrom. The mitigated pressure increase amount (third amount) in the lower mitigated pressure increase processing may be the same as the mitigated pressure increase amount (second amount) in the upper mitigated pressure increase processing, or may be different therefrom.

In the example described above, the CPU 112 uses the lower pressure increase mitigation threshold value and the upper pressure increase mitigation threshold value to switch the pressure increase processing. In place thereof, the CPU 112 may not use the upper pressure increase mitigation threshold value. In this case, the CPU 112 executes the lower mitigated pressure increase processing while the current treatment pressure is equal to or higher than the lowest treatment pressure but lower than the lower pressure increase mitigation threshold value. The CPU 112 executes the normal pressure increase processing while the current treatment pressure is equal to or higher than the lower pressure increase mitigation threshold value but lower than the highest treatment pressure.

In the method of FIG. 2 described above, the CPAP device 100 compares the current treatment pressure with the one threshold value (pressure reduction promotion threshold value), thereby selecting any one of the two types of pressure reduction processing (normal pressure reduction processing and promoted pressure reduction processing), and then executes the selected processing. In place thereof, the CPAP device 100 may compare the current treatment pressure with two or more threshold values, thereby selecting any one of two or more types of pressure reduction processing, and may then execute the selected processing. The two or more types of pressure reduction processing may include, in addition to the above-described normal pressure reduction processing and promoted pressure reduction processing, processing of reducing the treatment pressure more slowly than the normal pressure reduction processing.

For example, in a case in which the current treatment pressure is of a value close to the lowest treatment pressure, it is considered that the respiration disorder event has not occurred in the patient for a while. Thus, the CPAP device 100 executes the promoted pressure reduction processing while the current treatment pressure is of the value close to the lowest treatment pressure.

Specifically, while the current treatment pressure is equal to or higher than the lowest treatment pressure but lower than a lower pressure reduction promotion threshold value (fourth threshold value), the CPU 112 executes lower promoted pressure reduction processing. The CPU 112 executes the normal pressure reduction processing while the current treatment pressure is equal to or higher than the lower pressure reduction promotion threshold value but lower than an upper pressure reduction promotion threshold value (third threshold value). The CPU 112 executes upper promoted pressure reduction processing while the current treatment pressure is equal to or higher than the upper pressure reduction promotion threshold value but equal to or lower than the highest treatment pressure. The lower pressure reduction promotion threshold value and the upper pressure reduction promotion threshold value are each a threshold value to be compared with the current treatment pressure in order to switch the type of the pressure reduction processing. The lower pressure reduction promotion threshold value is smaller than the upper pressure reduction promotion threshold value. Each of the lower promoted pressure reduction processing and the upper promoted pressure reduction processing is processing of reducing the treatment pressure more quickly than the normal processing as in the promoted pressure reduction processing described before. The promoted pressure reduction condition (sixth condition) in the lower promoted pressure reduction processing may be the same as the promoted pressure reduction condition (fifth condition) in the upper promoted pressure reduction processing, or may be different therefrom. The promoted pressure reduction amount (sixth amount) in the lower promoted pressure reduction processing may be the same as the promoted pressure reduction amount (fifth amount) in the upper promoted pressure reduction processing, or may be different therefrom.

In the example described above, the CPU 112 uses the lower pressure reduction promotion threshold value and the upper pressure reduction promotion threshold value to switch the pressure reduction processing. In place thereof, the CPU 112 may not use the upper pressure reduction promotion threshold value. In this case, the CPU 112 executes the lower promoted pressure reduction processing while the current treatment pressure is equal to or higher than the lowest treatment pressure but lower than the lower pressure reduction promotion threshold value. The CPU 112 executes the normal pressure reduction processing while the current treatment pressure is equal to or higher than the lower pressure reduction promotion threshold value but equal to or lower than the highest treatment pressure.

In the method of FIG. 2 and the modification examples thereof described above, the CPAP device 100 selects and executes any one of the two or more types of the pressure increase processing (such as the normal pressure increase processing and the mitigated pressure increase processing), and selects and executes any one of the two or more types of the pressure reduction processing (such as the normal pressure reduction processing and the promoted pressure reduction processing). In place thereof, the CPAP device 100 may not switch the pressure increase processing (for example, the CPAP device 100 may always execute the normal pressure increase processing), and may execute any of the two types of pressure reduction processing. Further, the CPAP device 100 may not switch the pressure reduction processing (for example, the CPAP device 100 may always execute the normal pressure reduction processing), and may select any one of the two types of the pressure increase processing.

### <Plurality of Operation Modes>

The CPAP device 100 may operate in one operation mode selected from a plurality of operation modes including a standard mode and a soft mode. The soft mode (first operation mode) is an operation mode which focuses on adherence (continuity of the treatment). The standard mode (second operation mode) is an operation mode which focuses on a treatment effect. The CPAP device 100 increases the treatment pressure more slowly and reduces the treatment pressure more quickly in the soft mode than in the standard mode. The plurality of operation modes may include operation modes other than the standard mode and the soft mode. For example, the plurality of operation modes may include a fixed mode in which the treatment pressure is fixed to a value specified by the medical doctor.

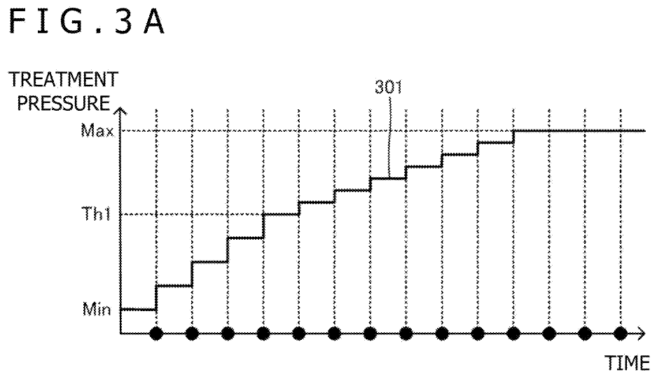

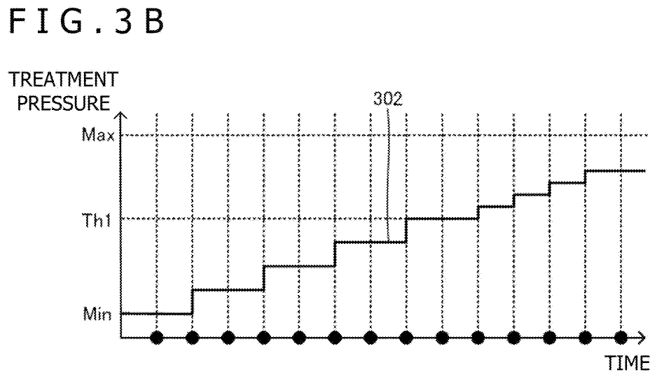

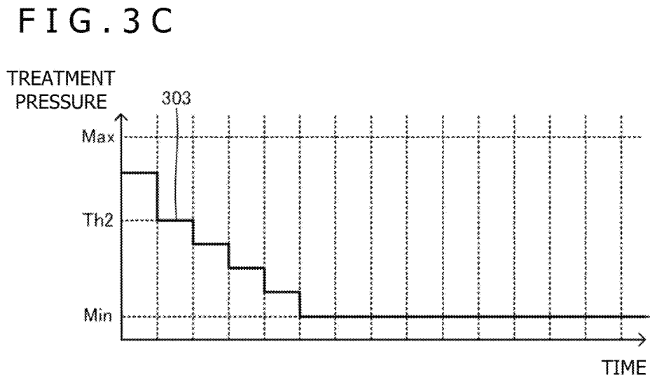

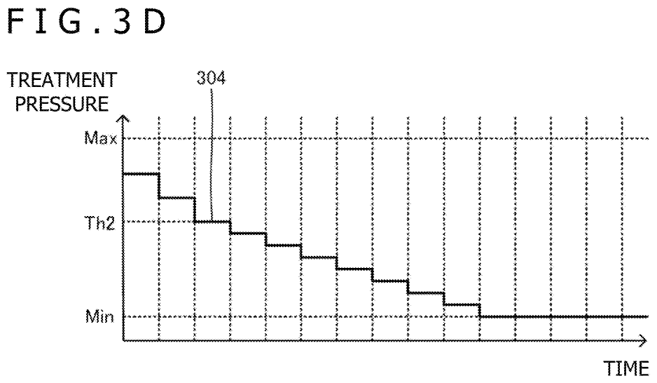

With reference to FIG. 3A to FIG. 3D, a description is now given of a change in treatment pressure in each of the standard mode and the soft mode. A graph 301 of FIG. 3A indicates a temporal change in treatment pressure at the time of the pressure increase in the standard mode. A graph 302 of FIG. 3B indicates a temporal change in treatment pressure at the time of the pressure increase in the soft mode. A graph 303 of FIG. 3C indicates a temporal change in treatment pressure at the time of the pressure reduction in the standard mode. A graph 304 of FIG. 3D indicates a temporal change in treatment pressure at the time of the pressure reduction in the soft mode. In each of the graphs 301 to 304, Max, Min, Th1, and Th2 indicate the highest treatment pressure, the lowest treatment pressure, the pressure increase mitigated threshold value, and the pressure reduction promotion threshold value, respectively. Each of black dots on a horizontal axis of each of the graph 301 and the graph 302 indicates a time at which the respiration disorder event occurred.

In the examples of FIG. 3A and FIG. 3B, the normal pressure increase amount, the mitigated pressure increase amount, and the pressure increase mitigation threshold value in the soft mode are the same as those in the standard mode, and the normal pressure increase condition and the mitigated pressure increase condition in the soft mode are less likely to be satisfied than those in the standard mode. Specifically, the normal pressure increase condition and the mitigated pressure increase condition in the soft mode are each such a condition that the respiration disorder event has been detected two times from the start of the supply of the air at the current treatment pressure. The normal pressure increase condition and the mitigated pressure increase condition in the standard mode are each such a condition that the respiration disorder event has been detected one time from the start of the supply of the air at the current treatment pressure. Thus, even when the respiration disorder event occurs in the patient in the same way, the treatment pressure increases more quickly in the standard mode than in the soft mode.

In the examples of FIG. 3C and FIG. 3D, the normal pressure reduction condition, the promoted pressure reduction condition, and the pressure reduction promotion threshold value in the soft mode are the same as those in the standard mode, and the normal pressure reduction amount and the promoted pressure reduction amount in the soft mode are smaller than those in the standard mode. Specifically, the normal pressure reduction condition and the promoted pressure reduction condition in both of the modes are each such a condition that the respiration disorder event is not detected for a predetermined period of time (for example, three minutes). The normal pressure reduction amount in the soft mode is a half of the normal pressure reduction amount in the standard mode. The promoted pressure reduction amount in the soft mode is a half of the promoted pressure reduction amount in the standard mode. Thus, even when the respiration disorder event is not detected for the same time, the treatment pressure decreases more quickly in the standard mode than in the soft mode.

The treatment pressure more slowly increases and more quickly decreases in the soft mode described above than in the standard mode. In place of the soft mode or in addition to the soft mode, the CPAP device 100 may be operable in a soft pressure reduction mode in which the treatment pressure more quickly decreases than in the standard mode and the treatment pressure increases similarly to the standard mode, and may be operable in a soft pressure increase mode in which the treatment pressure more slowly increases than in the standard mode and the treatment pressure decreases similarly to the standard mode.

### <Method of Changing Setting>

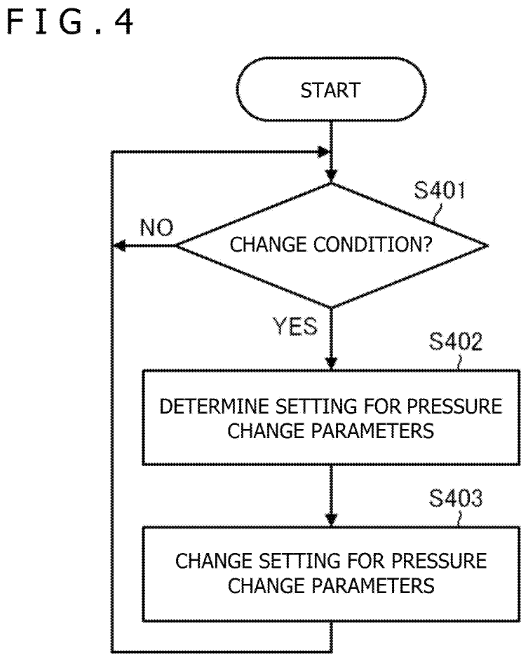

With reference to FIG. 4, a description is now given of a method executed by the CPAP device 100 to change a setting for treatment parameters. The treatment parameters include parameters relating to the treatment by the CPAP device 100. The treatment parameters include the highest treatment pressure, the lowest treatment pressure, pressure increase parameters, and pressure reduction parameters. The pressure increase parameters are parameters relating to the increase in treatment pressure in the method of FIG. 2, and are specifically the normal pressure increase amount, the mitigated pressure increase amount, the normal pressure increase condition, the mitigated pressure increase condition, and the pressure increase mitigation threshold value. The highest treatment pressure and the lowest treatment pressure are not included in the pressure increase parameters. The pressure reduction parameters are parameters relating to the reduction in treatment pressure in the method of FIG. 2, and are specifically the normal pressure reduction amount, the promoted pressure reduction amount, the normal pressure reduction condition, the promoted pressure reduction condition, and the pressure reduction promotion threshold value. The highest treatment pressure and the lowest treatment pressure are not included in the pressure reduction parameters. The pressure increase parameters and the pressure reduction parameters are generally referred to as pressure change parameters. A description given below relating to the pressure change parameters may be applied to both the pressure increase parameters and the pressure reduction parameters, may be applied to only the pressure increase parameters, or may be applied to only the pressure reduction parameters.

The CPAP device 100 may change the setting for all of the pressure change parameters, or may change the setting for some of them. As described later, the CPAP device 100 may change the setting for the pressure change parameters according to an instruction from the user (for example, the medical doctor), or may change the setting for them on the basis of an analysis result of the respiration disorder event. The CPAP device 100 may store a change history of the setting for the pressure change parameters. For example, the CPAP device 100 may store, in association with the setting after the change, at least one of whether or not this change has been made according to the instruction from the user, whether or not the this change has been made on the basis of the analysis result of the respiration disorder event, or a date and a time of the change.

The pressure change parameters changed by the CPAP device 100 are also referred to as changeable pressure change parameters. The pressure change parameters not changed by the CPAP device 100 are also referred to as unchangeable pressure change parameters. The unchangeable pressure change parameters are maintained to an initial setting made by the vendor of the CPAP device 100.

The CPAP device 100 may change the setting for the pressure change parameters for the soft mode, and may not change the setting for the pressure change parameters for the standard mode. In this case, the setting for the pressure change parameters for the standard mode is invariable, and is maintained to the initial setting made by the vendor of the CPAP device 100. The soft mode is the mode in which the adherence is focused upon, and hence, the setting for the pressure change parameters are changeable while usability of the CPAP device 100 felt by the patient is prioritized. Meanwhile, the standard mode is the mode in which the treatment effect is focused upon, and hence, the optimal setting for the treatment determined by the vendor of the CPAP device 100 is maintained. In place thereof, the CPAP device 100 may change the setting for the pressure change parameters also for the standard mode. In the following description, the change in setting for the pressure change parameters is reflected in the soft mode but not reflected in the standard mode (that is, the original setting is maintained). In place thereof, the CPAP device 100 may have the change in the pressure change parameters for the soft mode reflected also in the setting for the pressure change parameters for the standard mode. Further, the CPAP device 100 may change the setting for the pressure change parameters for the soft mode and the setting for the pressure change parameter for the standard mode independently of each other.

Each step of the method of FIG. 4 may be executed by the CPU 112 (for example, the setting change unit 122 thereof). Specifically, each step is executed by the CPU 112 executing the program read into the RAM 114. In place thereof, at least some of the steps of the method of FIG. 4 may be executed by a dedicated circuit such as an ASIC. The method of FIG. 4 may be started in response to such a state that a power supply of the CPAP device 100 is turned on, or may be started through another event as a trigger. The method of FIG. 4 may be ended in response to such a state that the power supply of the CPAP device 100 is turned off, or may be ended through another event as a trigger.

In S401, the CPU 112 determines whether or not a change condition is satisfied. In a case in which the CPU 112 determines that change condition is satisfied (“YES” in S401), the CPU 112 causes the processing to transition to S402, otherwise (“NO” in S401) the CPU 112 repeats S401. In this manner, the CPU 112 waits until the change condition is satisfied.

The change condition is a condition for changing the setting for the current pressure change parameters. The change condition may include such a condition that an instruction given by the medical doctor is acquired, may include such a condition that new treatment executed by the CPAP device 100 is to be started, and may include such a condition that a specific condition is satisfied during one time of the treatment or during a plurality of times of the treatment executed by the CPAP device 100.

In S402, the CPU 112 determines a new setting for the pressure change parameters. In S403, the CPU 112 changes the setting for the pressure change parameters such that the setting determined in S402 is brought about. Specifically, the CPU 112 updates the setting for the pressure change parameters stored in the RAM 114.

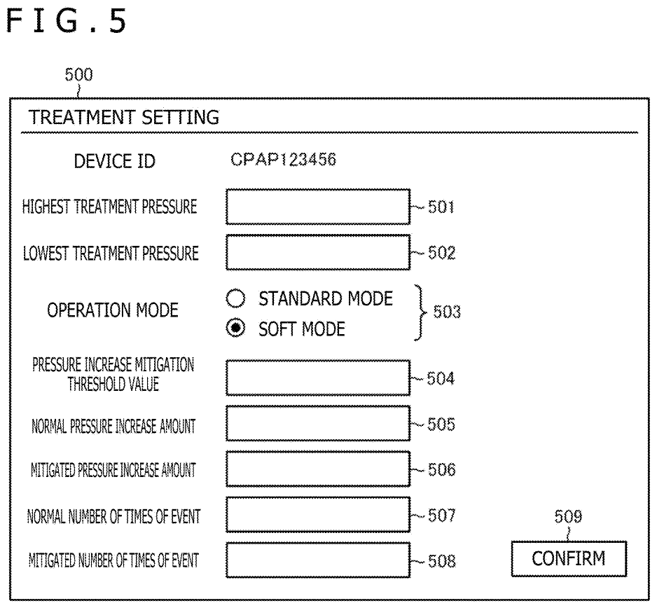

With reference to FIG. 5, a description is now given of the case in which the change condition includes the condition that the instruction given by the medical doctor is acquired. The external system 130 may generate a screen 500 for acquiring the instruction for changing the setting for the pressure change parameters and the new setting for the pressure change parameters from the medical doctor, and may provide a user interface including the screen 500. The screen 500 is displayed on a client terminal of the medical doctor connected to the external system 130.

The screen 500 may include objects illustrated in FIG. 5. An object 501 is an object (for example, a text box) for acquiring a specification for the highest treatment pressure from the medical doctor. An object 502 is an object (for example, a text box) for acquiring a specification for the lowest treatment pressure from the medical doctor. An object 503 is an object (for example, a radio button) for acquiring a specification for the operation mode of the CPAP device 100 from the medical doctor. In the example of the screen 500, any one of the standard mode and the soft mode can be specified.

An object 504 is an object (for example, a text box) for acquiring a specification for the pressure increase mitigation threshold value from the medical doctor. The value which can be input to the object 504 may be limited to a predetermined range (for example, 6 to 12 cmH₂O). An object 505 is an object (for example, a text box) for acquiring a specification for the normal pressure increase amount from the medical doctor. An object 506 is an object (for example, a text box) for acquiring a specification for the mitigated pressure increase amount from the medical doctor.

An object 507 is an object (for example, a text box) for acquiring a specification for the normal pressure increase condition from the medical doctor. Specifically, the object 507 acquires the required number of times of the respiration disorder event to be detected to satisfy the normal pressure increase condition. An object 508 is an object (for example, a text box) for acquiring a specification for the mitigated pressure increase condition from the medical doctor. Specifically, the object 508 acquires the required number of times of the respiration disorder event to be detected to satisfy the mitigated pressure increase condition.

The input to the objects 504 to 508 may be allowed only in the case in which the soft mode is selected on the object 503, or may not be allowed in the case in which the standard mode is selected on the object 503. On the screen 500, all of the pressure change parameters can be changed. In a case in which the change of some of the pressure change parameters is not allowed, the objects corresponding to these pressure change parameters are not displayed. The current setting for the pressure change parameters may be displayed on the objects 501 to 508 at a display start time of the screen 500.

An object 509 is an object (for example, a button) for acquiring, from the medical doctor, an instruction of having the setting for the objects 501 to 508 reflected in the CPAP device 100. In response to the pressing of the object 509, the external system 130 stores, in association with a specific CPAP device 100, the setting for the treatment parameters specified on the objects 501 to 508. The CPAP device 100 acquires a new setting for the treatment parameters from the external system 130 periodically or at a predetermined timing, and determines to use this setting as the new setting. After that, the CPAP device 100 updates the current setting for the treatment parameters stored in the RAM 114 such that this new setting is made.

In the example of FIG. 5, the screen 500 does not include objects for acquiring specifications for some (for example, the pressure reduction promotion threshold value, the promoted pressure reduction condition, and the promoted pressure reduction amount) of the pressure reduction parameters from the medical doctor, but the screen 500 may include these parameters. In place thereof, the CPAP device 100 may provide a screen for acquiring a specification for the setting for the pressure increase parameters and a screen for acquiring a specification for the setting for the pressure reduction parameters independently of each other.

The screen 500 may include the current setting for the pressure change parameters. The current setting for the pressure change parameters displayed on the screen 500 may be the setting relating to the mode selected on the object 503. In place thereof, the screen 500 may include the current setting for the pressure change parameters of each of the two modes. In this case, the user can compares the settings for the two modes with each other. The current setting for the standard mode and the current setting for the soft mode may be included in independent screens displayed side by side.

The CPAP device 100 may calculate a degree of a change in treatment pressure on the basis of the setting specified through use of the screen 500, and, for example, in a case in which the treatment pressure is determined to quickly increase or in a case in which the treatment pressure is determined to slowly decrease in the soft mode compared with the standard mode, may not accept this change in setting or may display a warning.

The screen 500 may be generated by the CPU 112 and displayed on the display unit 115. In this case, the medical doctor operates the operation unit 116 to specify the setting for the treatment parameters, and the CPU 112 determines the new setting for the treatment parameters according to this specification, and changes the current setting.

A description is now given of a case including the start of new treatment by the CPAP device 100. In the case in which the new treatment by the CPAP device 100 is started (that is, the patient starts to sleep), the CPU 112 may determine the new setting for the pressure change parameters of the patient for the soft mode on the basis of information measured by the CPAP device 100 during the sleep of the patient in the past. For example, the CPU 112 may determine the new setting for the pressure change parameters for the patient for the soft mode on the basis of the apnea-hypopnea index (AHI) (number of times of the apnea or the hypopnea per one hour) measured by the CPAP device 100 during the sleep of the patient for the previous time. Specifically, the CPU 112 may determine the new setting for the pressure change parameters such that, as the AHI is larger, the treatment pressure is more likely to increase (for example, the CPU 112 may increase the pressure increase mitigation threshold value and the pressure reduction promotion threshold value). In place of the AHI or in addition to the AHI, as the information measured by the CPAP device 100, there may be used a treatment time, the leak amount of the air from the mask 125, and the like.

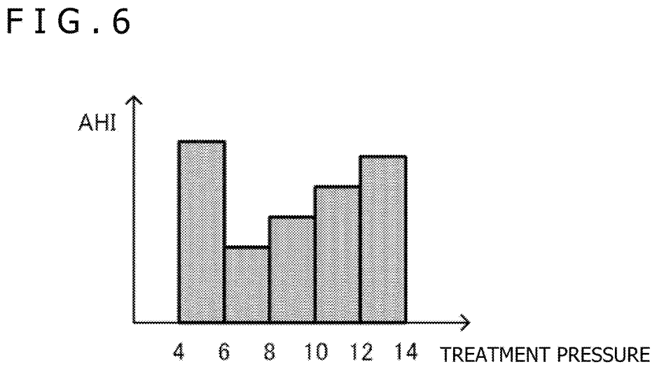

Further, the CPU 112 may analyze the information measured by the CPAP device 100 during the sleep of the patient in the past, thereby determining the new setting for the pressure change parameters. For example, as illustrated in FIG. 6, the CPU 112 indicates the AHI during the sleep of the patient for the previous time for each treatment pressure as a histogram. The CPU 112 may determine, as a new pressure increase mitigation threshold value, a treatment pressure which is among treatment pressures equal to or lower than a predetermined upper limit and at which a frequency of the AHI is the lowest.

In place thereof or in addition thereto, the CPU 112 may determine the new setting for the pressure change parameters for the soft mode on the basis of evaluation of the usability of the CPAP device 100 acquired from the patient. For example, the CPU 112 may determine the new setting for the pressure change parameters such that the treatment pressure is likely to increase (for example, the CPU 112 may increase the pressure increase mitigation threshold value and the pressure reduction promotion threshold value), on the basis of such evaluation that the treatment by the CPAP device 100 was comfortable. Meanwhile, the CPU 112 may determine the new setting for the pressure change parameters such that the treatment pressure is unlikely to increase (for example, the CPU 112 may reduce the pressure increase mitigation threshold value and the pressure reduction promotion threshold value), on the basis of such evaluation that the treatment by the CPAP device 100 was uncomfortable.

In place thereof or in addition thereto, the CPU 112 may determine the new setting for the pressure change parameters for the soft mode on the basis of attribute information of the patient. For example, the CPU 112 may determine the new setting for the pressure change parameters such that the treatment pressure is more likely to increase (for example, the CPU 112 may increase the pressure increase mitigation threshold value and the pressure reduction promotion threshold value) as a weight or a body mass index (BMI) of the patient increases.

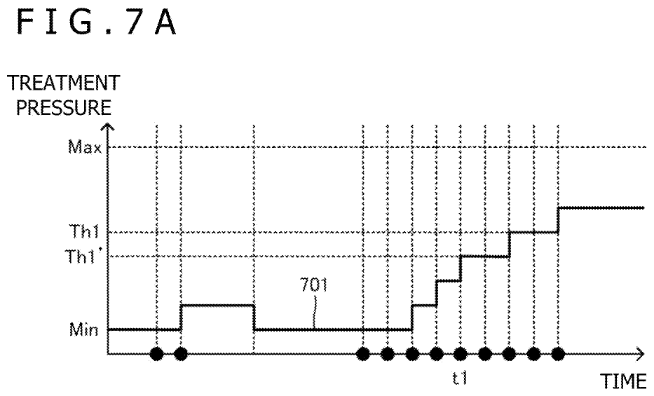

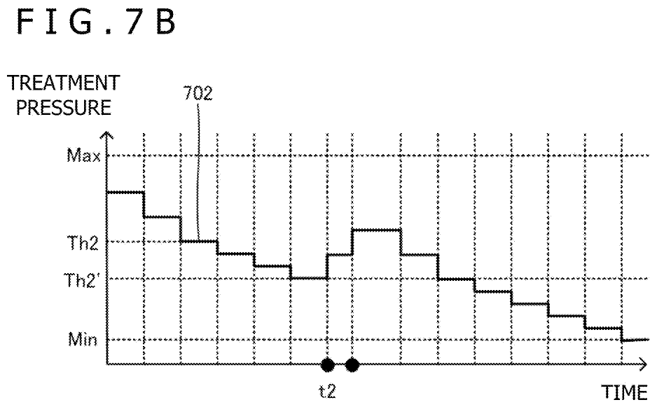

A description is now given of a case including satisfaction of a specific condition during the treatment executed once by the CPAP device 100. With reference to FIG. 7A, a description is now given of a change in treatment pressure in the soft mode. A graph 701 of FIG. 7A indicates a temporal change in treatment pressure in the soft mode. In the graph 701, Max, Min, Th1, and Th1′ indicate the highest treatment pressure, the lowest treatment pressure, the pressure increase mitigation threshold value before a change, and the pressure increase mitigation threshold value after the change, respectively. Each of black dots on a horizontal axis of the graph 701 indicates a time at which the respiration disorder event occurred.

The CPU 112 may determine the new setting for the pressure change parameters used during the sleep of the patient for this time for the soft mode, on the basis of the information measured by the CPAP device 100 during the sleep of the patient for this time. For example, in a case in which there has frequently been detected the respiration disorder event (for example, 20 times in the last 30 minutes) during the sleep of the patient for this time, the CPU 112 may determine, as a new pressure increase mitigation threshold value, the treatment pressure at that time or in a vicinity thereof. In FIG. 7A, it is assumed that this condition is satisfied at a time t1. Thus, the CPU 112 determines, as the new pressure increase mitigation threshold value, the treatment pressure Th1′ at that time or the vicinity thereof. In a case in which the respiration disorder events are frequently detected, the treatment pressure drastically increases in a short period of time. Thus, by changing the setting for the pressure change parameters such that the treatment pressure is less likely to increase, the possibility that the patient feels the sense of discomfort can be reduced.

In a case in which the number of times of detection of the central apnea during the sleep of the patient for this time is equal to or larger than a threshold value, the CPU 112 may determine, as the new pressure increase mitigation threshold value, a treatment pressure at that time or the vicinity thereof. In a case in which the number of times of the detection of the central apnea is large, even when the treatment pressure is increased, the apnea may not be mitigated. Thus, changing the setting for the pressure change parameters such that the treatment pressure is less likely to increase makes it possible to appropriately treat the patient.

With reference to FIG. 7B, a description is now given of a change in treatment pressure in the soft mode. A graph 702 of FIG. 7B indicates a temporal change in treatment pressure in the soft mode. In the graph 702, Max, Min, Th2, and Th2′ indicate the highest treatment pressure, the lowest treatment pressure, the pressure reduction promotion threshold value before a change, and the pressure reduction promotion threshold value after the change, respectively. Each of black dots on a horizontal axis of the graph 702 indicates a time at which the respiration disorder event occurred.

The CPU 112 may determine the new setting for the pressure change parameters used during the sleep of the patient for this time for the soft mode, on the basis of the information measured by the CPAP device 100 during the sleep of the patient for this time. For example, in a case in which a predetermined number of times of the respiration disorder event have continuously occurred during the pressure reduction of the treatment pressure, the CPU 112 may determine, as a new pressure reduction promotion threshold value, the treatment pressure at this time or in a vicinity thereof. In FIG. 7B, it is assumed that the predetermined number of times of the respiration disorder event have continuously occurred from a time t2 during the pressure reduction of the treatment pressure. Thus, the CPU 112 determines, as the new pressure reduction promotion threshold value, the treatment pressure Th2′ at that time or the vicinity thereof. As a result, the possibility that the patient feels the sense of discomfort can be reduced.

In the embodiment described above, the CPAP device 100 changes the setting for the pressure change parameters for the entire standard mode or for the entire soft mode. In place thereof or in addition thereto, the CPAP device 100 may change the setting for the pressure change parameters for each of types of the respiration disorder event (for example, the apnea, the hypopnea, the snore, and the flow limitation) being a detection target. For example, the CPAP device 100 may set, for the soft mode, the pressure increase mitigation threshold value to 10 cmH₂O for the apnea, and may set the pressure increase mitigation threshold value to 7 cmH₂O for the snore.

### <Provision of Information for Setting Pressure Change Parameters>

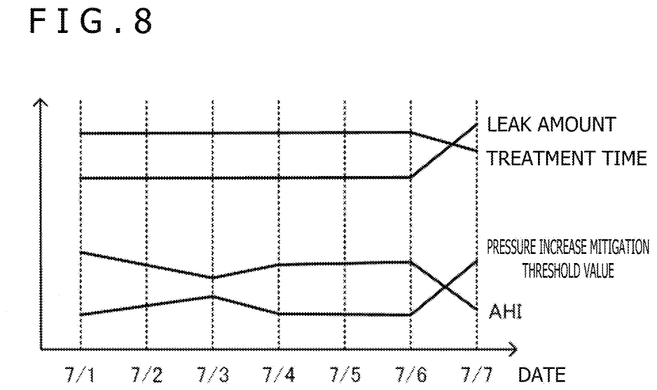

The CPAP device 100 may provide the medical doctor with information that allows the medical doctor to appropriately determine the setting for the pressure change parameters. For example, the CPU 112 may provide the medical doctor with information (for example, the treatment time, the leak amount of the air from the mask 125, and the AHI) measured by the CPAP device 100 during the sleep of the patient and the setting for the pressure change parameters (for example, the pressure increase mitigation threshold value) used during this sleep. The CPU 112 may provide these pieces of information to the external system 130. The external system 130 displays these pieces of information in a form as illustrated in FIG. 8 for the medical doctor. The medical doctor may refer to this graph to determine the setting for the pressure change parameters (for example, the pressure increase mitigation threshold value). The graph of FIG. 8 may be displayed for the medical doctor by the display unit 115.

### Second Embodiment

The snore causes a high-frequency component to be generated in a respiratory waveform. However, a cause of the high-frequency component generated in the respiratory waveform is not limited to the snore. The present embodiment identifies a cause of the high-frequency component included in the respiratory waveform and executes processing corresponding to the cause, thereby increasing continuity of the treatment while avoiding the discomfort about the use of the CPAP device felt by the patient.

### <Configuration of Cpap Device>

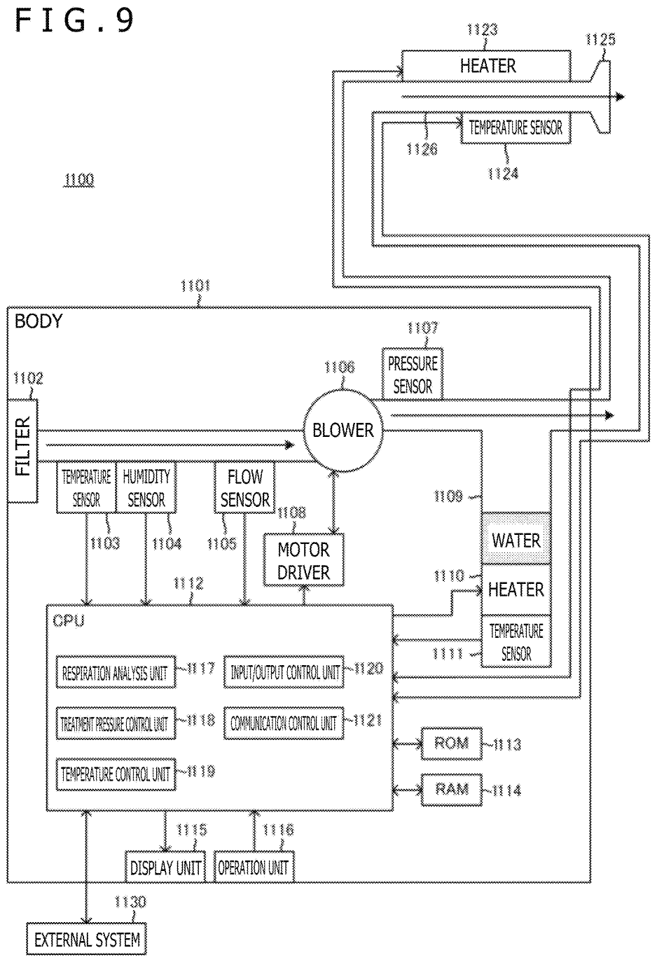

With reference to FIG. 9, a description is now given of a configuration example of a continuous positive airway pressure device (hereinafter referred to as the CPAP device) 1100 according to some embodiments. The CPAP device 1100 is an example of the medical device which treats the SAS. The medical device is a device used for the medicine. For example, the medical device is a device used for surgery, treatment, or diagnosis. The CPAP device 1100 has a snore detection function. The CPAP device 1100 includes a body 1101, a mask 1125, and a tube 1126 which connects the body 1101 and the mask 1125 to each other. An operation of the CPAP device 1100 is implemented by a CPU 1112 reading a program stored in a ROM 1113 into a RAM 1114 and then executing the program. A device including a processor such as the CPU 1112 and a memory such as the ROM 1113 and the RAM 1114 as described above may be considered as a computer. Note that functional blocks 1117 to 1121 illustrated in the CPU 1112 are principal functions which are among the various functions implemented by the CPU 1112 executing the program and which are expressed schematically. Thus, an operation described as being performed by each of the functional blocks 1117 to 1121 is, in practice, implemented by the CPU 1112 executing the program. In place thereof, one or more functional blocks may be implemented by a hardware circuit other than the CPU 1112.

First, a description is given of components that are present in a flow passage of air. A filter 1102 is provided at an inlet opening of the air, and removes pollens, bacteria, dusts, and the like. A temperature sensor 1103 measures a temperature of the air which has flown in. A measurement value obtained by the temperature sensor 1103 is supplied to a temperature control unit 1119. A humidity sensor 1104 measures humidity of the air which has flown in. A measurement value obtained by the humidity sensor 1104 is supplied to the temperature control unit 1119.

A flow (differential pressure) sensor 1105 (hereinafter simply referred to as a flow sensor 1105) is, for example, the flow rate sensor of the differential pressure type, and measures the flow rate of the air in the flow passage on the basis of a pressure difference between an upstream side and a downstream side. Here, it is assumed that there is acquired a positive measurement value in the case in which the pressure on the upstream side is higher than the pressure on the downstream side and there is acquired a negative measurement value in the case in which the pressure on the downstream side is higher than the pressure on the upstream side. Thus, it is possible to also recognize the flow direction of the air in the flow passage from the measurement value of the flow sensor 1105. The measurement value of the flow sensor 1105 is supplied to a respiration analysis unit 1117.

A blower 1106 internally includes an impeller and a motor that drives the impeller. A treatment pressure control unit 1118 controls a rotation speed of the motor via a motor driver 1108. A flow rate and a supply pressure of the air supplied to the airway of the patient can thereby be adjusted.

A pressure sensor 1107 is provided downstream of the blower 1106, and measures a pressure in the flow passage. A measurement value of the pressure sensor 1107 is supplied to the treatment pressure control unit 1118. The treatment pressure control unit 1118 controls a supply pressure while assuming that the air is supplied to the airway of the patient at the pressure measured by the pressure sensor 1107.

A humidifier 1109 includes a water storage tank, and humidifies the air supplied to the tube 1126. Here, the temperature control unit 1119 controls a temperature of a heater 1110 provided to the humidifier 1109, thereby controlling an amount of the water vaporized from the water storage tank, that is, a degree of the humidification. A temperature sensor 1111 measures a temperature of the heater 1110, and supplies the measured temperature to the temperature control unit 1119. In the present embodiment, the heater 1110 is used for the humidifier 1109 to humidify the air supplied to the airway of the patient as well as executing temperature adjustment. Note that the temperature adjustment for the air can be implemented through another method such as using, for example, a heater 1123 provided to the tube 1126. In the case in which the humidification and the temperature adjustment are executed independently of each other, the heater 1110 and the temperature sensor 1111 may not be provided to the humidifier 1109. Moreover, for example, the air may be blown to a water surface in the water storage tank, or the flow passage may be disposed in such a manner as to pass through the water.

The tube 1126 connects the body 1101 and the mask 1125 to each other. The tube 1126 has an elastic property and a bending property in order to be able to easily follow a movement of the mask 1125. A temperature sensor 1124 which measures a temperature of the air supplied to the airway of the patient is provided to the tube 1126. A measurement value of the temperature sensor 1124 is supplied to the temperature control unit 1119.

The mask 1125 has such a size and a shape as to cover the nose and the mouth of the patient, and is worn by the patient through use of a string or band adjustable in length. For example, a supply unit which supplies the air toward the airway of the patient at the treatment pressure includes the blower 1106, the mask 1125, and the tube 1126.

A display unit 1115 is, for example, a display provided to a housing of the body 1101, and displays a message relating to handling of the CPAP device 1100, various menu screens used to set the CPAP device 1100, measurement values of various sensors, and the like. The display on the display unit 1115 is controlled by an input/output control unit 1120.

An operation unit 1116 is a general name of input devices operable by the user, for example, buttons and switches provided to the housing of the body 1101. In a case in which the display unit 1115 is a touch display, the display unit 1115 and the operation unit 1116 are integrally formed. An operation on the operation unit 1116 is detected by the input/output control unit 1120, and the CPU 1112 executes an operation corresponding to the detected operation.

The respiration analysis unit 1117 detects an occurrence of an event defined in advance, on the basis of the flow rate measured by the flow sensor 1105. When detecting the occurrence of the event defined in advance, the respiration analysis unit 1117 notifies the treatment pressure control unit 1118 of the occurrence of the event. The respiration analysis unit 1117 may be able to detect an occurrence of the snore, the flow limitation, the apnea and the hypopnea, and the Cheyne-Stokes respiration of the patient. Further, the respiration analysis unit 1117 may be able to detect a leak amount of the air from the mask 1125.

The treatment pressure control unit 1118 controls, on the basis of the measurement value of the pressure sensor 1107, an operation of the blower 1106 to supply the air to the airway of the patient such that the supply air pressure is the treatment pressure. Moreover, the treatment pressure control unit 1118 controls the supply air pressure in response to the notification from the respiration analysis unit 1117. For example, the treatment pressure control unit 1118 switches, in response to the notification from the respiration analysis unit 1117, between control of the treatment pressure at the inspiration time of the patient and control of the treatment pressure at the expiration time. The treatment pressure control unit 1118 provides, to the motor driver 1108, for example, a duty ratio of a pulse voltage applied to the motor, to control the rotation speed of the impeller of the blower 1106, thereby controlling the supply air pressure. The treatment pressure control unit 1118 notifies the temperature control unit 1119 of the supply air pressure set currently.

The temperature control unit 1119 controls the operation of the heater 1110 according to the measurement values of the temperature sensor 1103, the humidity sensor 1104, the temperature sensor 1111, and the temperature sensor 1124 and the supply air pressure the notification of which is given from the treatment pressure control unit 1118, thereby controlling the temperature and the humidity of the air supplied to the airway of the patient. The temperature and the humidity of the air supplied to the airway of the patient may be a temperature and humidity set by the user via the operation unit 1116. The temperature control unit 1119 notified of the user setting made via the operation unit 1116, via, for example, the input/output control unit 1120. The supply air pressure is taken into consideration because a degree of an increase in temperature is low in a case in which the flow rate is high compared with a case in which the flow rate is low even when the temperature of the heater 1110 is constant.

A communication control unit 1121 executes processing relating to communication between the body 1101 and an external system 1130. The communication control unit 1121 is compliant with, for example, one or more publicly-known wireless and/or wired communication standards and is able to execute the communication with the external system 1130. The external system 1130 may be, for example, a management system for examination and treatment data in a hospital or a remote management system for the CPAP device 1100. A medical system includes the CPAP device 1100 and the external system 1130.

### <Operation Method for CPAP Device>

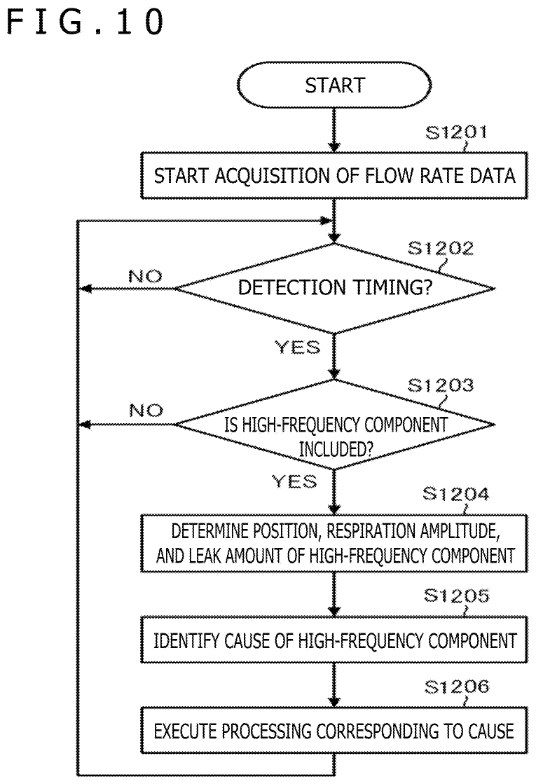

With reference to FIG. 10 to FIG. 12, a description is now given of a method of causing the CPAP device 1100 to operate. In this method, the cause of the high-frequency component included in the respiratory waveform is identified as any one of a plurality of causes including the snore. In a case in which the cause of the high-frequency component is identified as the snore, an occurrence of the snore is to be detected. The snore is possibly vibration sound generated from the epipharynx when the air passes through the airway which is narrowed during the sleep of the patient. Further, in this method, there is executed processing corresponding to the cause of the high-frequency component. In the following description, each step of the method of FIG. 10 is executed by the CPU 1112 (for example, the respiration analysis unit 1117 or the treatment pressure control unit 1118 thereof). Specifically, each step is executed by the CPU 1112 executing the program read into the RAM 1114. In place thereof, at least some of the steps of FIG. 10 may be executed by a dedicated circuit such as an ASIC. The method of FIG. 10 may be started in response to such a state that start of an operation (for example, a treatment operation during the sleep) of the CPAP device 1100 is instructed by the patient, may be started through, as a trigger, the CPAP device 1100 detecting such a state that the patient falls asleep, or may be started through another event as a trigger. The method of FIG. 10 may be ended in response to such a state that end of the operation (for example, the treatment operation during the sleep) of the CPAP device 1100 is instructed by the patient, may be ended through, as a trigger, the CPAP device 1100 detecting such a state that the patient gets out of bed, or may be ended through another event as a trigger.

In S1201, the CPU 1112 starts acquisition of flow rate data. The value measured by the flow sensor 1105 is continued to be supplied to the CPU 1112 (for example, the respiration analysis unit 1117 thereof) during the execution of the method of FIG. 10. As described above, the value measured by the flow sensor 1105 indicates the flow rate of the respiration of the patient during the sleep. The CPU 1112 samples the flow rate supplied by the flow sensor 1105, at a predetermined sampling interval (for example, 2 ms), and stores, as flow rate data, the flow rate at each time in the RAM 1114. This flow rate data indicates the flow rate of the respiration of the patient as a time series. As now described in detail, the CPU 1112 analyzes this flow rate data, thereby detecting the occurrence of the hypopnea. The CPU 1112 may apply a low-pass filter to the flow rate data (for example, the CPU 1112 may calculate a moving average of the flow rate), thereby removing a noise component and a snore component when the flow rate data is generated. The flow rate data is an example of time-series data relating to the respiration (that is, respiration data) of the patient. In the method of FIG. 10, in place of the flow rate data, there may be used other types of respiration data, for example, pressure difference data indicating a time series of the pressure difference of the air flowing through the tube 1126. In the description of FIG. 10 given below, the flow rate data the acquisition of which is started in S1201 is simply referred to as flow rate data.

After the acquisition of the flow rate data is started in S1201, the CPU 1112 determines, in S1202, whether or not the current timing is a timing for detecting the occurrence of the snore. In a case in which the CPU 1112 determines that the current timing is the timing for detecting the occurrence of the snore (“YES” in S1202), the CPU 1112 causes the processing to transition to S1203, otherwise (“NO” in S1202) the CPU 1112 repeats S1202. In a case in which the subsequent steps S1202 to S1206 are periodically (for example, every 30 seconds) executed, the CPU 1112 may determine that the current timing is the timing for detecting the occurrence of the snore, on the basis of elapse of the predetermined period of time (for example, 30 seconds) from determination of the timing for detecting the occurrence of the snore for the previous time.

In S1203, the CPU 1112 determines whether or not an analysis target portion of the respiratory waveform indicated by the flow rate data the acquisition of which is started in S1201 includes a high-frequency component satisfying a predetermined snore detection condition. In a case in which the CPU 1112 determines that the analysis target portion of the respiratory waveform includes the high-frequency component satisfying the predetermined condition (“YES” in S1203), the CPU 1112 causes the processing to transition to S120l , otherwise (“NO” in S1203) the CPU 1112 causes the processing to transition to S1202.

The analysis target portion of the respiratory waveform is a portion of the respiratory waveform to be analyzed for determining whether or not the snore is occurring. For example, the analysis target portion is a portion of the respiratory waveform included in a period having the predetermined time length (for example, 30 seconds) an end point of which is the execution time of S1202. The snore detection condition in S1203 is a condition which can be used to detect the occurrence of the snore. In general, the snore has a frequency equal to or higher than 10 Hz. Thus, the snore detection condition may be such a condition that a spectrum (a peak or an area) of a frequency component equal to or higher than 10 Hz is equal to or more than a threshold value.

Specifically, the CPU 1112 may apply frequency analysis to the analysis target portion. For example, the CPU 1112 applies signal processing including the Fourier transform to the analysis target portion of the flow rate data, thereby generating the frequency data. This signal processing may include filtering before the Fourier transform. The Fourier transform may be executed through the fast Fourier transform (FFT). After that, the CPU 1112 compares the frequency data with reference data in the spectrum equal to or higher than 10 Hz. The reference data is data which includes frequency distributions, and includes a frequency distribution which the frequency data can take in a case in which it is assumed that the snore is not occurring. The reference data is, for example, frequency data on an analysis target portion which is included in the analysis target portion when S1203 was executed before and which is determined not to satisfy the snore detection condition. In a case in which the analysis target portion at the time when S1203 was executed before does not exist, data created in advance may be used as the reference data. The snore detection condition may be such a condition that, in a frequency band equal to or higher than 10 Hz, an integrated value of a difference between the frequency data and the reference data in a band in which the frequency data exceeds the reference data is equal to or larger than a threshold value. In place thereof or in addition thereto, the snore detection condition may be such a condition that the maximum value of values each obtained by subtracting the reference data from the frequency data is equal to or larger than a threshold value in the frequency band equal to or higher than 10 Hz.

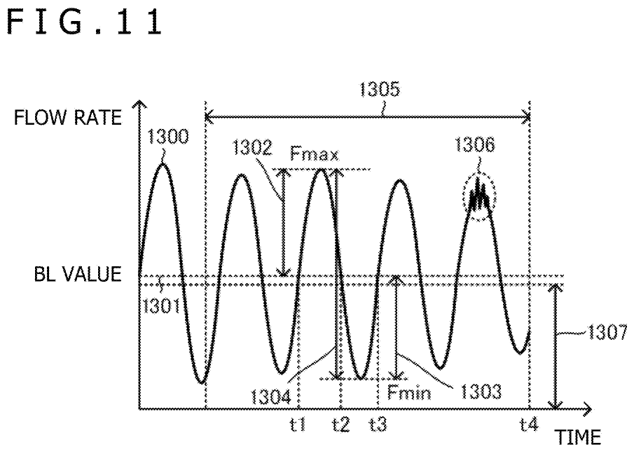

In S1204, the CPU 1112 determines a position of the high-frequency component of the analysis target portion, a respiration amplitude of the analysis target portion, and the leak amount of the analysis target portion. With reference to FIG. 11, a description is now given of a specific example of the processing in S1204. A graph 1300 of FIG. 11 indicates a part of the respiratory waveform indicated by the flow rate data acquired up to a time t4. A horizontal axis of the graph 1300 of FIG. 11 indicates time, and a vertical axis of the graph 1300 indicates the flow rate of the respiration of the patient at each time. A baseline 1301 being a center of the amplitude of the respiratory waveform is also indicated. A flow rate corresponding to the baseline 1301 is indicated as a baseline (BL) value. The BL value may be constant over the sleep of the patient, or may temporally change. The BL value may be updated for each respiration, or may be, for example, an average value of the flow rate in the last predetermined period (for example, 10 seconds) of each respiration.

One time of vibration of the respiratory waveform indicates one time of the respiration. A start point and an end point of the one time of the respiration may be set as desired. In the description given below, a start of the inspiration of the patient is set to the start of the one time of the respiration, and an end of the expiration of the patient is set to the end of the one time of the respiration. In place thereof, a start of the expiration of the patient may be set to the start of the one time of the respiration, and an end of the inspiration of the patient may be set to the end of the one time of the respiration.

A period in which the one time of the respiration is executed is referred to as a respiration period (for example, a time t1 to a time t3). A period which is included in the respiration period and in which the inspiration is executed is referred to as an inspiration period (for example, the time t1 to the time t2). The inspiration period is a period in which the flow rate data is larger than the BL value. A period which is included in the respiration period and in which the expiration is executed is referred to as an expiration period (for example, the time t2 to the time t3). The expiration period is a period in which the flow rate data is smaller than the BL value.

The respiration amplitude is an index indicating a magnitude of the one time of the respiration. For example, the respiration amplitude may be a difference between a maximum value Fmax and the BL value of the flow rate in the one time of the respiration period (the time t1 to the time t3). This value is referred to as an inspiration amplitude 1302. In place thereof, the respiration amplitude may be a difference between a minimum value Fmin of the flow rate and the BL value in the one time of the respiration period (the time t1 to the time t3). This value is referred to as an expiration amplitude 1303. In place thereof, the respiration amplitude may be a difference between the maximum value Fmax and the minimum value Fmin in the one time of the respiration period (the time t1 to the time t3). This value is referred to as a peak-to-peak value 1304. The CPU 1112 determines, as the respiration amplitude of the analysis target portion 1305, a representative value (for example, the average value, a median, the maximum value, or the minimum value) of the respiration amplitude of one or more times of the respiration included in the analysis target portion 1305. Moreover, the CPU 1112 determines an average value of the flow rate in the analysis target portion 1305 as a leak amount 1307 of the air supplied toward the airway of the patient in the analysis target portion 1305.

Further, the CPU 1112 determines whether or not a portion (high-frequency portion 1306) including the high-frequency component satisfying the snore detection condition in S1203 is included in the inspiration portion of any respiration in the analysis target portion 1305. In the example of FIG. 11, the high-frequency portion 1306 is included in the inspiration portion. The state in which the high-frequency portion 1306 is included in the inspiration portion may be a state in which the high-frequency portion 1306 is completely included in the inspiration portion (that is, the high-frequency portion 1306 does not overlap with a portion other than the inspiration portion), or may be a state in which the high-frequency portion 1306 is partially included in the inspiration portion (that is, a part of the high-frequency portion 1306 overlaps with the portion other than the inspiration portion).

In S1205, the CPU 1112 identifies, in response to the determination of the various values in S1204, a cause of the high-frequency component included in the analysis target portion 1305 among the plurality of causes on the basis of determination criteria. The plurality of causes may include the snore. The plurality of causes may further include at least one of noise caused by a leak of the air supplied toward the airway of the patient, obstruction of the tube 1126 which supplies the air toward the airway of the patient, or impact applied to the tube 1126. For example, the plurality of causes may include all of them. The determination criteria may be based on whether or not the inspiration portion of the respiratory waveform includes the high-frequency portion 1306. The determination criteria may further be based on at least one of the respiration amplitude of the analysis target portion 1305 or the leak amount 1307 of the air supplied toward the airway of the patient. For example, the determination criteria may be based on all of them.

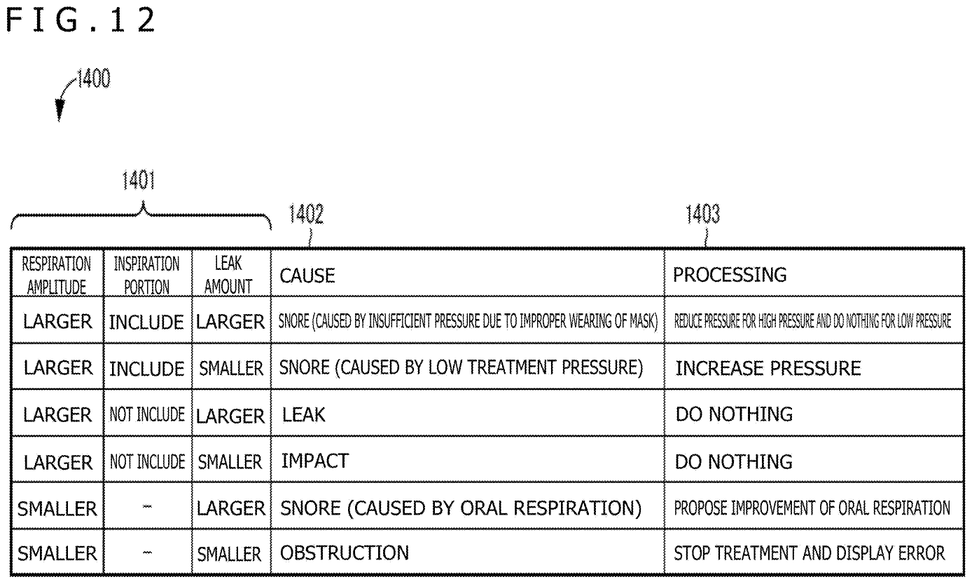

In response to the identification of the cause of the high-frequency component included in the analysis target portion 1305 in S1205, the CPU 1112 executes processing corresponding to this cause in S1206. With reference to a table 1400 of FIG. 12, determination criteria 1401, a cause 1402 of the high-frequency component, and processing 1403 corresponding to the cause 1402. In an example of the table 1400, the determination criteria 1401 are based on the respiration amplitude of the analysis target portion 1305 (“respiration amplitude” in FIG. 12), whether or not the inspiration portion of the respiratory waveform includes the high-frequency portion 1306 (“inspiration portion” of FIG. 12), and the leak amount 1307 of the air supplied to the airway of the patient (“leak amount” of FIG. 12).

The determination criterion relating to the respiration amplitude may be such a criterion that the respiration amplitude of the analysis target portion 1305 is equal to or larger than an amplitude threshold value (“larger” in FIG. 12) or smaller than the amplitude threshold value (“smaller” in FIG. 12). The amplitude threshold value for the respiration amplitude may be a fixed value or a value obtained by multiplying a representative value (for example, an average value) of the past respiration amplitudes by a coefficient (for example, 0.3 or 1). The determination criterion relating to the inspiration portion may be whether the inspiration portion of the analysis target portion 1305 includes (“include” in FIG. 12) or does not include (“not include” in FIG. 12) the high-frequency portion 1306. In a case in which the respiration amplitude of the analysis target portion 1305 is smaller than the amplitude threshold value, there is such a possibility that the inspiration portion is not identified accurately. Thus, in the example of table 1400, in a case in which the respiration amplitude of the analysis target portion 1305 is smaller than the amplitude threshold value, whether or not the inspiration portion of the analysis target portion 1305 includes the high-frequency portion 1306 is not included in the determination criteria. The determination criterion relating to the leak amount 1307 may be whether the leak amount 1307 of the analysis target portion 1305 is equal to or larger than a leak threshold value (“larger” in FIG. 12) or smaller than the leak threshold value (“smaller” in FIG. 12). The leak threshold value for the leak amount 1307 may be a fixed value (for example, 70 L/min) or a value obtained by multiplying a representative value (for example, an average value) of the past leak amounts by a coefficient (for example, 1.5 or 1).

In the example of the table 1400, the cause 1402 includes the snore, the noise caused by the leak of the air supplied toward the airway of the patient (“leak” of FIG. 12), and the obstruction of the tube 1126 (“obstruction” of FIG. 12). Further, in the case in which the cause is the snore, a detailed cause thereof is described in the table 1400. In a case in which the respiration amplitude is large and the inspiration portion includes the high-frequency portion 1306, the CPU 1112 identifies the cause of the high-frequency component as the snore. Moreover, also in a case in which the respiration amplitude is small and the leak amount 1307 is large, the CPU 1112 identifies the cause of the high-frequency component as the snore.

In a case in which the respiration amplitude is large, the inspiration portion does not include the high-frequency portion 1306, and the leak amount 1307 is large, the CPU 1112 identifies the cause of the high-frequency component as the noise caused by the leak of the air supplied toward the patient. In a case in which the respiration amplitude is large, the inspiration portion does not include the high-frequency portion 1306, and the leak amount 1307 is small, the CPU 1112 identifies the cause of the high-frequency component as the impact applied to the tube 1126. In a case in which the respiration amplitude is small, and the leak amount 1307 is small, the CPU 1112 identifies the cause of the high-frequency component as the obstruction of the tube 1126.

A description is now given of the processing 1403 executed by the CPU 1112 for each cause. The CPU 1112 controls the treatment pressure on the basis of the cause of the high-frequency component. In a case in which the cause of the high-frequency component is identified as the snore, the CPU 1112 controls the treatment pressure on the basis of how the determination criterion 1401 is satisfied. The cause of the snore identified in the case in which the respiration amplitude is large, the inspiration portion includes the high-frequency portion 1306, and the leak amount 1307 is large is considered as such a cause that the mask 1125 is not correctly worn by the patient and hence the air at a sufficient pressure is not supplied to the airway of the patient. Thus, the CPU 1112 reduces the treatment pressure when the treatment pressure is equal to or higher than a pressure threshold value (for example, 10 cmH₂O). A displacement of the mask 1125 can be dissolved by reducing the treatment pressure in this way. On the other hand, the CPU 1112 does not change the treatment pressure on the basis of the cause of the high-frequency component when the treatment pressure is lower than the pressure threshold value (for example, 10 cmH₂O). Even in the case in which the change in treatment pressure based on the cause of the high-frequency component is not made, the CPU 1112 may change the treatment pressure in response to satisfaction of another condition (for example, the detection of the occurrence of the apnea or the hypopnea). The same applies to a case in which the change in treatment pressure based on the cause of the high-frequency component in a description given below is not made.

The cause of the snore identified in the case in which the respiration amplitude is large, the inspiration portion includes the high-frequency portion 1306, and the leak amount 1307 is small is considered as such a cause that the treatment pressure is low. Thus, the CPU 1112 increases the treatment pressure. The cause of the snore identified through the state in which the respiration amplitude is small and the leak amount 1307 is large is considered as such a cause that the patient is executing oral respiration. Thus, the CPU 1112 does not change the treatment pressure on the basis of the cause of the high-frequency component. Further, the CPU 1112 may propose improvement of the oral respiration to the patient or the medical doctor. Specifically, the CPU 1112 may propose to change the mask 1125 to a mask of a full-face type, or to sleep while the mouth is closed by a tape.

In a case in which the cause of the high-frequency component is identified as the noise caused by the leak or the impact on the tube 1126, the CPU 1112 does not make the change in treatment pressure based on the cause of the high-frequency component, and does not execute particular processing. In a case in which the cause of the high-frequency component is identified as the obstruction of the tube 1126, the CPU 1112 does not make the change in treatment pressure based on the cause of the high-frequency component, stops the treatment, and displays an error on the display unit 1115.

In the embodiment described above, the method of FIG. 10 is executed by the CPAP device 1100. In place thereof, the method of FIG. 10 may be executed by a test device for the SAS. Such a test device as described above does not have the function of supplying the compressed air to the patient. The above-described detection of the hypopnea may be executed by a medical device such as an adaptive servo ventilation (ASV) device, a home oxygen therapy (HOT) device, and an artificial respirator.

In the embodiment described above, the occurrence of the hypopnea in the patient is detected in real time during the sleep of the patient. In place thereof, the method of FIG. 10 may be executed after the patient gets out of bed, by using flow rate data acquired during the sleep of the patient. That is, the occurrence of the hypopnea in the patient may be detected after the patient gets out of bed. As a result, an occurrence period of the snore can be identified and can hence be used for the diagnosis of the patient.

In the embodiment described above, the method of FIG. 10 is executed by the CPAP device 1100. In place thereof, the method of FIG. 10 may be executed by a medical device included in the external system 1130. Specifically, the medical device included in the external system 1130 is configured to use the data (for example, the flow rate data) measured by the CPAP device 1100 to execute the method of FIG. 10. In this case, in S1206, the medical device included in the external system 1130 causes the CPAP device 1100 to execute processing corresponding to the cause.

According to the present disclosure, the following configurations can be provided.

### (Item 1)

A medical device including:
a supplier configured to supply air to an airway of a patient at a treatment pressure; and
a processor,
in which the processor is configured to
   change the treatment pressure by a first amount in a case in which a first condition relating to respiration disorder is satisfied while the treatment pressure is lower than a first threshold value,
   change the treatment pressure by a second amount in a case in which a second condition relating to the respiration disorder is satisfied while the treatment pressure is equal to or higher than the first threshold value, and
   change a setting for a pressure change parameter including at least one of the first amount, the second amount, the first condition, the second condition, or the first threshold value for a first operation mode of the medical device.

With this item, it is possible to change the pressure change parameter such that the discomfort of the patient is mitigated. Thus, the continuity of treatment for the patient increases.

### (Item 2)

The medical device according to Item 1,
changing the treatment pressure includes increasing the treatment pressure,
wherein the processor is configured to
   increase the treatment pressure by the first amount in a case in which the first condition relating to respiration disorder is satisfied while the treatment pressure is lower than the first threshold value,
   increase the treatment pressure by the second amount in a case in which the second condition relating to the respiration disorder is satisfied while the treatment pressure is equal to or higher than the first threshold value, and
   change a setting for a pressure increase parameter including at least one of the first amount, the second amount, the first condition, the second condition, or the first threshold value for the first operation mode of the medical device.

With this item, it is possible to change the pressure increase parameter such that the discomfort of the patient is mitigated. Thus, the continuity of treatment for the patient increases.

### (Item 3)

The medical device according to Item 2,
in which the processor is configured to
   increase the treatment pressure by the first amount in a case in which the first condition is satisfied while the treatment pressure is equal to or higher than a second threshold value but lower than the first threshold value, and
   increase the treatment pressure by a third amount in a case in which a third condition relating to the respiration disorder is satisfied while the treatment pressure is lower than the second threshold value.

With this item, the treatment pressure can finely be changed.

### (Item 4)

The medical device according to Item 2,
in which the processor is configured to
   reduce the treatment pressure by a fourth amount in a case in which a fourth condition relating to the respiration disorder is satisfied while the treatment pressure is lower than a third threshold value,
   reduce the treatment pressure by a fifth amount in a case in which a fifth condition relating to the respiration disorder is satisfied while the treatment pressure is equal to or higher than the third threshold value, and
   change a setting for a pressure reduction parameter including at least one of the fourth amount, the fifth amount, the fourth condition, the fifth condition, or the third threshold value for the first operation mode.

With this item, it is possible to change the pressure reduction parameter such that the discomfort of the patient is mitigated. Thus, the continuity of treatment for the patient increases.

### (Item 5)

The medical device according to Item 1,
changing the treatment pressure includes reducing the treatment pressure,
wherein the processor is configured to
   reduce the treatment pressure by the first amount in a case in which the first condition relating to respiration disorder is satisfied while the treatment pressure is lower than the first threshold value,
   reduce the treatment pressure by the second amount in a case in which the second condition relating to the respiration disorder is satisfied while the treatment pressure is equal to or higher than the first threshold value, and
   change a setting for a pressure reduction parameter including at least one of the first amount, the second amount, the first condition, the second condition, or the first threshold value for the first operation mode of the medical device.

With this item, it is possible to change the pressure reduction parameter such that the discomfort of the patient is mitigated. Thus, the continuity of treatment for the patient increases.

### (Item 6)

The medical device according to Item 5,
reduce the treatment pressure by the first amount in a case in which the first condition is satisfied while the treatment pressure is equal to or higher than a second threshold value but lower than the first threshold value, and
reduce the treatment pressure by a third amount in a case in which a third condition relating to the respiration disorder is satisfied while the treatment pressure is lower than the second threshold value.

With this item, the treatment pressure can finely be changed.

### (Item 7)

The medical device according to Item 5,
in which the processor is configured to determine a new setting for the pressure change parameter for the first operation mode on the basis of at least one of
   information measured by the medical device during past sleep of the patient,
   attribute information of the patient,
   evaluation of usability of the medical device acquired from the patient, or
   information measured by the medical device during current sleep of the patient.

With this item, it is possible to appropriately set the pressure change parameter.

### (Item 8)

The medical device according to Item 1,
in which the processor is configured to maintain the setting for the pressure change parameter for a second operation mode different from the first operation mode when the pressure change parameter for the first operation mode is changed.

With this item, it is possible to set the medical device to the mode in which the treatment efficiency is prioritized.

### (Item 9)

A non-transitory storage medium that stores a program for making a computer execute a method of operating a medical device including a supplier configured to supply air to an airway of a patient at a treatment pressure, the method including
changing the treatment pressure by a first amount in a case in which a first condition relating to respiration disorder is satisfied while the treatment pressure is lower than a first threshold value,
changing the treatment pressure by a second amount in a case in which a second condition relating to the respiration disorder is satisfied while the treatment pressure is equal to or higher than the first threshold value, and
changing a setting for a parameter including at least one of the first amount, the second amount, the first condition, the second condition, or the first threshold value for a first operation mode of the medical device.

With this item, it is possible to change the treatment change parameter such that the discomfort of the patient is mitigated. Thus, the continuity of treatment for the patient increases.

### (Item 10)

A medical device including:
a processor configured to
   detect that an analysis target portion of a respiratory waveform of a patient includes a high-frequency component that satisfies a predetermined condition, and
   identify, on the basis of a determination criterion, a cause of the high-frequency component among a plurality of causes,
in which the plurality of causes include a snore, and
the determination criterion is based on whether or not an inspiration portion of the respiratory waveform includes the high-frequency component.

With this item, it is possible to identify the cause of the high-frequency component included in the respiratory waveform. Thus, it is possible to execute the appropriate processing corresponding thereto.

### (Item 11)

The medical device according to Item 10,
in which the determination criterion is further based on at least one of a respiration amplitude of the analysis target portion or a leak amount of air supplied toward an airway of the patient.

With this item, it is possible to accurately identify the cause of the high-frequency component.

### (Item 12)

The medical device according to Item 10,
in which the plurality of causes further include at least one of noise caused by a leak of air supplied toward an airway of the patient, impact applied to a tube that supplies the air toward the airway of the patient, or obstruction of the tube.

With this item, it is possible to identify the specific cause as described above.

### (Item 13)

The medical device according to Item 10, further including:
a supplier configured to supply air toward an airway of the patient at a treatment pressure,
in which the processor controls the treatment pressure on the basis of the cause of the high-frequency component.

With this item, it is possible to use a medical device such as the CPAP device to identify the cause of high-frequency component included in the respiratory waveform.

### (Item 14)

The medical device according to Item 13,
in which the processor increases the treatment pressure on the basis of such a condition that a leak amount of the air supplied toward the airway of the patient is smaller than a leak threshold value in a case in which the cause of the high-frequency component is identified as the snore.

With this item, the treatment pressure can appropriately be increased.

### (Item 15)

The medical device according to Item 13,
in which the processor, on the basis of such a condition that a leak amount of the air supplied toward the airway of the patient is equal to or larger than a leak threshold value and that a respiration amplitude of the analysis target portion is smaller than an amplitude threshold value in a case in which the cause of the high-frequency component is identified as the snore, avoids a change in treatment pressure based on the cause of the high-frequency component.

With this item, an excessive increase in treatment pressure can be suppressed.

### (Item 16)

The medical device according to Item 13,
in which the processor, on the basis of such a condition that a leak amount of the air supplied toward the airway of the patient is equal to or larger than a leak threshold value and that a respiration amplitude of the analysis target portion is equal to or larger than an amplitude threshold value in a case in which the cause of the high-frequency component is identified as the snore, reduces the treatment pressure when the treatment pressure is equal to or higher than a pressure threshold value and avoids a change in treatment pressure based on the cause of the high-frequency component when the treatment pressure is lower than the pressure threshold value.

With this item, the treatment pressure can appropriately be increased.

### (Item 17)

The medical device according to Item 13,
in which the processor, in a case in which the cause of the high-frequency component is identified as one of noise caused by a leak of the air supplied toward the airway of the patient, impact applied to a tube that supplies the air toward the airway of the patient, or obstruction of the tube, avoids change in treatment pressure based on the cause of the high-frequency component.

With this item, an increase in treatment pressure due to erroneous detection of the snore can be suppressed.

### (Item 18)

A non-transitory storage medium that stores a program for making a computer execute a method of operating a medical device, the method including
detecting that an analysis target portion of a respiratory waveform of a patient includes a high-frequency component that satisfies a predetermined condition, and
identifying, on the basis of a determination criterion, a cause of the high-frequency component among a plurality of causes,
in which the plurality of causes include a snore, and
the determination criterion is based on whether or not an inspiration portion of the respiratory waveform includes the high-frequency component.

With this item, it is possible to identify the cause of the high-frequency component included in the respiratory waveform. Thus, it is possible to execute the appropriate processing corresponding thereto.

The present disclosure is not limited to the embodiments described above and can be modified and changed in various ways within the gist of the disclosure.

What is claimed is:

1. A medical device comprising:
a supplier configured to supply air to an airway of a patient at a treatment pressure; and
a processor,
wherein the processor is configured to
change the treatment pressure by a first amount in a case in which a first condition relating to respiration disorder is satisfied while the treatment pressure is lower than a first threshold value,
change the treatment pressure by a second amount in a case in which a second condition relating to the respiration disorder is satisfied while the treatment pressure is equal to or higher than the first threshold value, and
change a setting for a pressure change parameter including at least one of the first amount, the second amount, the first condition, the second condition, or the first threshold value for a first operation mode of the medical device.

2. The medical device according to claim 1,
changing the treatment pressure includes increasing the treatment pressure,
wherein the processor is configured to
increase the treatment pressure by the first amount in a case in which the first condition relating to respiration disorder is satisfied while the treatment pressure is lower than the first threshold value,
increase the treatment pressure by the second amount in a case in which the second condition relating to the respiration disorder is satisfied while the treatment pressure is equal to or higher than the first threshold value, and
change a setting for a pressure increase parameter including at least one of the first amount, the second amount, the first condition, the second condition, or the first threshold value for the first operation mode of the medical device.

3. The medical device according to claim 2,
wherein the processor is configured to
increase the treatment pressure by the first amount in a case in which the first condition is satisfied while the treatment pressure is equal to or higher than a second threshold value but lower than the first threshold value, and
increase the treatment pressure by a third amount in a case in which a third condition relating to the respiration disorder is satisfied while the treatment pressure is lower than the second threshold value.

4. The medical device according to claim 2,
wherein the processor is configured to
reduce the treatment pressure by a fourth amount in a case in which a fourth condition relating to the respiration disorder is satisfied while the treatment pressure is lower than a third threshold value,
reduce the treatment pressure by a fifth amount in a case in which a fifth condition relating to the respiration disorder is satisfied while the treatment pressure is equal to or higher than the third threshold value, and
change a setting for a pressure reduction parameter including at least one of the fourth amount, the fifth amount, the fourth condition, the fifth condition, or the third threshold value for the first operation mode.

5. The medical device according to claim 1,
changing the treatment pressure includes reducing the treatment pressure,
wherein the processor is configured to
reduce the treatment pressure by the first amount in a case in which the first condition relating to respiration disorder is satisfied while the treatment pressure is lower than the first threshold value,
reduce the treatment pressure by the second amount in a case in which the second condition relating to the respiration disorder is satisfied while the treatment pressure is equal to or higher than the first threshold value, and
change a setting for a pressure reduction parameter including at least one of the first amount, the second amount, the first condition, the second condition, or the first threshold value for the first operation mode of the medical device.

6. The medical device according to claim 5,
reduce the treatment pressure by the first amount in a case in which the first condition is satisfied while the treatment pressure is equal to or higher than a second threshold value but lower than the first threshold value, and
reduce the treatment pressure by a third amount in a case in which a third condition relating to the respiration disorder is satisfied while the treatment pressure is lower than the second threshold value.

7. The medical device according to claim 1,
wherein the processor is configured to determine a new setting for the pressure change parameter for the first operation mode on a basis of at least one of
information measured by the medical device during past sleep of the patient,
attribute information of the patient,
evaluation of usability of the medical device acquired from the patient, or
information measured by the medical device during current sleep of the patient.

8. The medical device according to claim 1,
wherein the processor is configured to maintain the setting for the pressure change parameter for a second operation mode different from the first operation mode when the pressure change parameter for the first operation mode is changed.

9. A non-transitory storage medium that stores a program for making a computer execute a method of operating a medical device including a supplier configured supply air to an airway of a patient at a treatment pressure, the method including
changing the treatment pressure by a first amount in a case in which a first condition relating to respiration disorder is satisfied while the treatment pressure is lower than a first threshold value,
changing the treatment pressure by a second amount in a case in which a second condition relating to the respiration disorder is satisfied while the treatment pressure is equal to or higher than the first threshold value, and
changing a setting for a parameter including at least one of the first amount, the second amount, the first condition, the second condition, or the first threshold value for a first operation mode of the medical device.

10. A medical device comprising:
a processor configured to
detect that an analysis target portion of a respiratory waveform of a patient includes a high-frequency component that satisfies a predetermined condition, and
identify, on a basis of a determination criterion, a cause of the high-frequency component among a plurality of causes,
wherein the plurality of causes include a snore, and
the determination criterion is based on whether or not an inspiration portion of the respiratory waveform includes the high-frequency component.

11. The medical device according to claim 10,
wherein the determination criterion is further based on at least one of a respiration amplitude of the analysis target portion or a leak amount of air supplied toward an airway of the patient.

12. The medical device according to claim 10,
wherein the plurality of causes further include at least one of noise caused by a leak of air supplied toward an airway of the patient, impact applied to a tube that supplies the air toward the airway of the patient, or obstruction of the tube.

13. The medical device according to claim 10, further comprising:
a supplier configured to supply air toward an airway of the patient at a treatment pressure,
wherein the processor controls the treatment pressure on a basis of the cause of the high-frequency component.

14. The medical device according to claim 13,
wherein the processor increases the treatment pressure on a basis of such a condition that a leak amount of the air supplied toward the airway of the patient is smaller than a leak threshold value in a case in which the cause of the high-frequency component is identified as the snore.

15. The medical device according to claim 13,
wherein the processor, on a basis of such a condition that a leak amount of the air supplied toward the airway of the patient is equal to or larger than a leak threshold value and that a respiration amplitude of the analysis target portion is smaller than an amplitude threshold value in a case in which the cause of the high-frequency component is identified as the snore, avoids a change in treatment pressure based on the cause of the high-frequency component.

16. The medical device according to claim 13,
wherein the processor, on a basis of such a condition that a leak amount of the air supplied toward the airway of the patient is equal to or larger than a leak threshold value and that a respiration amplitude of the analysis target portion is equal to or larger than an amplitude threshold value in a case in which the cause of the high-frequency component is identified as the snore, reduces the treatment pressure when the treatment pressure is equal to or higher than a pressure threshold value and avoids a change in treatment pressure based on the cause of the high-frequency component when the treatment pressure is lower than the pressure threshold value.

17. The medical device according to claim 13,
wherein the processor, in a case in which the cause of the high-frequency component is identified as one of noise caused by a leak of the air supplied toward the airway of the patient, impact applied to a tube that supplies the air toward the airway of the patient, or obstruction of the tube, avoids change in treatment pressure based on the cause of the high-frequency component.
